(19) United States
(12) Patent Application Publication
(10) Pub. No.: US 2022/0220554 A1
(43) Pub. Date: Jul. 14, 2022

(54) SINGLE-GENE SINGLE-BASE RESOLUTION RATIO DETECTION METHOD FOR RNA CHEMICAL MODIFICATION

(71) Applicant: PEKING UNIVERSITY, Beijing (CN)

(72) Inventors: Guifang JIA, Beijing (CN); Yu XIAO, Beijing (CN)

(21) Appl. No.: 17/281,357

(22) PCT Filed: Sep. 30, 2018
(86) PCT No.: PCT/CN2018/109145

§ 371 Date: Mar. 30, 2021

(51) Int. Cl. C12Q 1/6876; C12Q 1/6827; C12Q 1/6851; C12Q 1/26; C12Q 1/48

(52) U.S. Cl. CPC C12Q 1/6876; C12Q 1/6827; C12Q 1/6851; C12Q 1/26; C12Q 2600/166; C12Q 1/48; C12Y 201/01; C12Q 2600/154; C12Y 114/11

(57) ABSTRACT

Provided is a method for detecting the chemical modification of a target RNA site X, comprising the steps as follows: (1) acquiring an RNA sample and selecting in the RNA sample a target RNA segment comprising the target RNA site X; (2) SELECT; (3) PCR amplification; (4) comprising the PCR cycle threshold value with a reference PCR cycle threshold value, or comparing the PCR amplification product quantity with a reference PCR amplification product quantity, so as to determine whether there is a target chemical modification in the target RNA site X. Further provided are a method for identifying a substrate target site of RNA modification enzyme or RNA demodification enzyme and a method for quantifying an RNA modification rate in a transcript.

### FIELD OF THE INVENTION

The present disclosure relates to the field of molecular biology, and in particular to a single-gene single-base resolution detection method for RNA chemical modification.

### BACKGROUND OF THE INVENTION

Over one hundred types of chemical modifications to RNA have been found among three domains of life, i.e., bacteria, archaebacteria, and eukaryote. The epitranscriptomic mark N⁶-methyladenosine (m⁶A) is the most abundant post-transcriptional RNA modification in both eukaryotic mRNA and long non-coding RNA (lncRNA). These marks are commonly installed by an m⁶A modification enzyme, and several sub-units of human m⁶A modification enzymes (methyltransferase complexes) have been identified: METTL3, METTL14, WTAP, KIAA1429 and RBM15 (RNA binding motif protein 15). The m⁶A located between MAT2A hairpin and spliceosome U6 snRNA is introduced by METTL16. The m⁶A is erased by AlkB family dioxygenases (e.g., FTO and ALKBH5 in human), which is referred as demodification enzyme. The m⁶A-binding proteins can read the m⁶A marks. It is known that m⁶A marks can regulate RNA processing and metabolism, including precursor mRNA splice, nuclear export, mRNA stability and translation. Therefore, m⁶A marks play a role in the adjustment in many biological processes such as stem cell differentiation, circadian rhythm, ultraviolet-induced DNA injury and disease pathogenesis.

Up to now, the transcriptomic detection method of m⁶A depends on m⁶A-antibody immunoprecipitation (m⁶A-IP), which is mainly attributed to the inert reactivity of methyl in m⁶A. The first developed method, m⁶A-sequencing (or MeRIP-seq), combines m⁶A-IP and high-throughput sequencing to locate the m⁶A sites within the RNA segments of about 200 nucleotides. Subsequently, m⁶A researchers developed PA-m⁶A-seq and miCLIP methods to map m⁶A marks at a higher resolution. Specifically, PA-m⁶A-seq method incorporates 4-thiouridine (45 U) in vivo, so as to crosslink the anti-m⁶A antibody with RNA under the exposure of UV (365 nm), thereby locating m⁶A site at about 23 nucleotides of resolution; miCLIP method crosslinks RNA with anti-m⁶A antibody under the exposure of UV (254 nm), and may identify m⁶A residue at single nucleotide resolution based on reverse transcription-induced mutation or truncation. Owing to issues with the specificity and low crosslinking yields of the anti-m⁶A antibodies, PA-m⁶A-seq or miCLIP methods can only identify a limited subset of the m⁶A sites, and neither method is widely used in m⁶A studies like m⁶A/MeRIP-seq.

Although m⁶A sequencing provides transcriptomic-wide information, a method for detecting specific m⁶A modifications of single transcripts is highly desirable for the studies of the biological functions of m⁶A. The m⁶A-IP-qPCR method is widely used in the study of function of m⁶A. However, it does not provide single base resolution, and cannot quantify, and it depends on the specificity of the m⁶A antibody. Several methods have been developed to detect m⁶A marks at single nucleotide resolution. To date, the RNase H-based SCARLET method is the only one that can quantitatively detect m⁶A status of single mRNA or lncRNA locus but its time-consuming nature and the need for radioactive labeling have limited its wider application.

### SUMMARY OF THE INVENTION

The present application provides a method for detecting a target chemical modification of an RNA target site X, comprising:

(1) obtaining an RNA sample, and selecting a target RNA segment containing an RNA target site X in the RNA sample;

(2) SELECT step: designing an up probe Px1 and a down probe Px2 for an upstream sequence and a downstream sequence of the RNA target site X within the target RNA segment, respectively, elongating the down probe Px2 through a DNA polymerase to obtain an elongated down probe Px2, and ligating the up probe Px1 and the elongated down probe Px2 through a ligase to obtain a SELECT product;

wherein, the up probe Px1 is complementary paired with the upstream sequence of the RNA target site X, and the first nucleotide of 5′-terminal of the up probe Px1 is complementary paired with a nucleotide located at a site with a distance of 1 nt from the RNA target site X at the upstream sequence of the RNA target site X;

the down probe Px2 is complementary paired with the downstream sequence of the RNA target site X, and the first nucleotide of 3′-terminal of the down probe Px2 is complementary paired with a nucleotide located at a site with a distance of 1, 2, 3, 4, 5, 6, 7, 8, 9 or 10 nt from the RNA target site X at the downstream sequence of the RNA target site X;

preferably, a length of sequence of the up probe Px1 that is complementary paired with the upstream sequence of the RNA target site X is 15-30 lit; a length of sequence of the down probe Px2 that is complementary paired with the downstream sequence of the RNA target site X is 15-30 nt;

(3) PCR amplification step: performing PCR amplification of the SELECT product obtained in step (2), determining a threshold cycle of PCR or an amount of PCR amplification product, preferably determining the threshold cycle of PCR by qPCR fluorescence signal, or preferably determining the amount of PCR amplification product by polyacrylamide gel electrophoresis; and

(4) comparing the threshold cycle of PCR to a threshold cycle of PCR reference, or comparing the amount of PCR amplification product to an amount of PCR amplification product reference, to determine if the target chemical modification is present at the RNA target site X.

In some embodiments of the present application, the chemical modification is selected from the group consisting of m⁶A modification, m¹A modification, pseudouridine modification, and 2′-O-methylation modification.

In some embodiments of the present application, the DNA polymerase is Bst 2.0 DNA polymerase or Tth DNA polymerase, preferably Bst 2.0 DNA polymerase; and the ligase is selected from the group consisting of SplintR ligase, T3 DNA ligase, T4 RNA ligase 2, and T4 DNA ligase, preferably SplintR ligase or T3 DNA ligase.

In some embodiments of the present application, in step (4), the threshold cycle of PCR reference is a threshold cycle of first PCR reference or a threshold cycle of second PCR reference, wherein:

the threshold cycle of first PCR reference is:

a threshold cycle of PCR of a first reference sequence determined by a method as same as that of the target RNA segment, wherein the first reference sequence comprises at least a nucleotide sequence II, and the nucleotide sequence II comprises a nucleotide sequence sharing a same nucleotide sequence with a nucleotide sequence I in the target RNA segment, wherein the nucleotide sequence I is a nucleotide sequence from a nucleotide that is complementary paired with a nucleotide of 3′-terminal of the up primer of the site X to a nucleotide that is complementary paired with a nucleotide of 5′-terminal of the down primer of the site X in the RNA target segment, and no target chemical modification is present in an RNA target site X1 of the first reference sequence corresponding to the RNA target site X of the target RNA segment; or

the threshold cycle of second PCR reference is:

a threshold cycle of PCR of a second reference sequence determined by a method as same as that of the target RNA segment, wherein the second reference sequence comprises at least a nucleotide sequence II, and the nucleotide sequence II comprises a nucleotide sequence sharing a same nucleotide sequence with a nucleotide sequence I in the target RNA segment, wherein the nucleotide sequence I is a nucleotide sequence from a nucleotide that is complementary paired with a nucleotide of 3′-terminal of the up primer of the site X to a nucleotide that is complementary paired with a nucleotide of 5′-terminal of the down primer of the site X in the RNA target segment, and the target chemical modification is present in an RNA target site X2 of the second reference sequence corresponding to the RNA target site X of the target RNA segment.

It should be noted that when “sharing a same nucleotide sequence” is mentioned in the text, the modification on the nucleotide is not considered. That is, the modification status or the modification types of two RNAs sharing the same nucleotide sequence can be same or different.

In some embodiments of the present application, when the threshold cycle of PCR is more than the threshold cycle of first PCR reference, it is determined that the target chemical modification is present in the RNA target site X; or

when the threshold cycle of PCR is equal to the threshold cycle of second PCR reference, it is determined that the target chemical modification is present in the RNA target site X.

In some embodiments of the present application, when the threshold cycle of PCR is at least 0.4-10 cycles, preferably at least 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10 cycles more than the threshold cycle of first PCR reference, it is determined that the target chemical modification is present at the RNA target site X.

In some embodiments of the present application, when the threshold cycle of PCR is more than the threshold cycle of first PCR reference by at least 0.4-10 cycles, preferably at least 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10 cycles, it is determined that the target chemical modification is present in the RNA target site X.

In some embodiments of the present application, in step (4), the amount of PCR amplification product reference is an amount of first PCR amplification product reference or an amount of PCR second amplification product reference, wherein:

the amount of first PCR amplification product reference is:

an amount of PCR amplification product of a first reference sequence determined by a method as same as that of the target RNA segment, wherein the first reference sequence comprises at least a nucleotide sequence II, and the nucleotide sequence II comprises a nucleotide sequence sharing a same nucleotide sequence with a nucleotide sequence I in the target RNA segment, wherein the nucleotide sequence I is a nucleotide sequence from a nucleotide that is complementary paired with a nucleotide of 3′-terminal of the up probe Px1 of the site X to a nucleotide that is complementary paired with a nucleotide at 5′-terminal of the down probe Px2 of the site X in the RNA target segment, and no target chemical modification is present in an RNA target site X1 of the first reference sequence corresponding to the RNA target site X of the target RNA segment; or

the amount of second PCR amplification product reference is:

an amount of PCR amplification product of a second reference sequence determined by a method as same as that of the target RNA segment, wherein the second reference sequence comprises at least a nucleotide sequence II, and the nucleotide sequence II comprises a nucleotide sequence sharing a same nucleotide sequence with a nucleotide sequence I in the target RNA segment, wherein the nucleotide sequence I is a nucleotide sequence from a nucleotide that is complementary paired with a nucleotide of 3′-terminal of the up probe Px1 of the site X to a nucleotide that is complementary paired with a nucleotide of 5′-terminal of the down probe Px2 of the site X in the RNA target segment, and the target chemical modification is present in an RNA target site X2 of the second reference sequence corresponding to the RNA target site X of the target RNA segment.

In some embodiments of the present application, when the amount of PCR amplification product is less than the amount of first PCR amplification product reference, it is determined that the target chemical modification is present in the RNA target site X; or

when the amount of PCR amplification product is equal to the amount of second PCR amplification product reference, it is determined that the target chemical modification is present in the RNA target site X.

In some embodiments of the present application, the method further comprises following steps:

(c) controlling initial RNA input amounts, randomly selecting an RNA non-target site N in the target RNA segment, preferably, the RNA non-target site N is located from 6^{th} nt of the upstream sequence of the RNA target site X to 2^{nd} nt of the downstream sequence of the RNA target site X; designing an up probe Pn1 and a down probe Pn2 for an upstream sequence and a downstream sequence of the RNA non-target site N, respectively, elongating the down probe Pn2 through a DNA polymerase to obtain an elongated down probe Pn2, and ligating the up probe Pn1 and the elongated down probe Pn2 through a ligase to obtain a SELECT product;

performing PCR amplification of the SELECT product, and determining a threshold cycle of PCR;

controlling the initial RNA input amounts of the target RNA segment according to the threshold cycle of PCR, so that the initial RNA input amounts of the target RNA segment is equal to initial RNA input amounts of a first reference sequence or a second reference sequence;

wherein,

the first reference sequence comprises at least a nucleotide sequence II, and the nucleotide sequence II comprises a nucleotide sequence sharing a same nucleotide sequence with a nucleotide sequence I in the target RNA segment, wherein when the site N is located upstream of the site X, the nucleotide sequence I is a nucleotide sequence from a nucleotide that is complementary paired with a nucleotide of 3′-terminal of the up probe Pn1 of the site N to a nucleotide that is complementary paired with a nucleotide of 5′-terminal of the down probe Px2 of the site X in the target RNA segment; when the site N is located downstream of the site X, the nucleotide sequence I is a nucleotide sequence from a nucleotide that is complementary paired with a nucleotide of 3′-terminal of the up probe Px1 of the site X to a nucleotide that is complementary paired with a nucleotide of 5′-terminal of the down probe Pn2 of the site N in the target RNA segment; and no target chemical modification is present in an RNA target site X1 of the first reference sequence corresponding to the RNA target site X of the target RNA segment; or

the second reference sequence comprises at least a nucleotide sequence II, and the nucleotide sequence II comprises a nucleotide sequence sharing a same nucleotide sequence with a nucleotide sequence I in the target RNA segment, wherein when the site N is located upstream of the site X, the nucleotide sequence I is a nucleotide sequence from a nucleotide that is complementary paired with a nucleotide of 3′-terminal of the up probe Pn1 of the site N to a nucleotide that is complementary paired with a nucleotide of 5′-terminal of the down probe Px2 of the site X in the target RNA segment, when the site N is located downstream of the site X, the nucleotide sequence I is a nucleotide sequence from a nucleotide that is complementary paired with a nucleotide of 3′-terminal of the up probe Px1 of the site X to a nucleotide that is complementary paired with a nucleotide of 5′-terminal of the down probe Pn2 of the site N in the target RNA segment; and target chemical modification is present in an RNA target site X2 of the second reference sequence corresponding to the RNA target site X of the target RNA segment.

In some embodiments of the present application, the SELECT step is performed in a reaction system comprising:

an RNA sample, preferably the RNA sample is a total RNA or mRNA extracted from cells; more preferably, a concentration of the total RNA or mRNA is 10 ng, 1 ng, 0.2 ng, 0.02 ng or lower; or more preferably, the concentration of the total RNA or mRNA is 10 ng, 100 ng, 1 μg, 10 μg or higher;

dNTP, preferably dTTP, more preferably 5-100 μM of dTTP;

a DNA polymerase, preferably Bst 2.0 DNA polymerase, more preferably 0.0005-0.05 U of Bst 2.0 DNA polymerase, most preferably 0.01 U of Bst 2.0 DNA polymerase;

a ligase, preferably SplintR ligase, more preferably 0.1-2 U of SplintR ligase, most preferably 0.5 U of SplintR ligase. In some embodiments of the present application, the SELECT step is performed at a reaction temperature of 30-50° C., preferably 37-42° C., more preferably 40° C.

In some embodiments of the present application, the method further comprises following step prior to the step (1):

treating the RNA sample with an RNA demodification enzyme or a mixture of the RNA demodification enzyme and EDTA, respectively; wherein the RNA sample treated with the RNA demodification enzyme is used as a first reference sequence;

preferably, the RNA demodification enzyme is FTO or ALKBH5.

In some embodiments of the present application, the RNA sample is total RNA, mRNA, rRNA, or lncRNA extracted from cells.

The present application also provides a method for identifying a target site of an RNA modification enzyme or an RNA demodification enzyme, comprising:

(1) preparing RNA modification enzyme—deficient or RNA demodification enzyme—deficient cells, or RNA modification enzyme—low expressed or RNA demodification enzyme—low expressed cells, culturing the cells and extracting an RNA after culturing the cells;

(2) determining a threshold cycle of PCR or an amount of PCR amplification product for an RNA target site X according to the above steps (1)-(3);

(3) comparing the threshold cycle of PCR with a threshold cycle of PCR reference, or comparing the amount of PCR amplification product with an amount of PCR amplification product reference, to determine if a chemical modification is performed by the RNA modification enzyme or the RNA demodification enzyme at the RNA target site X,

wherein, the threshold cycle of PCR reference is a threshold cycle of PCR for a normal cell determined by a method as same as that of the RNA modification enzyme—deficient or the RNA demodification enzyme—deficient cells, or the RNA modification enzyme—low expressed or the RNA demodification enzyme—low expressed cells,

the amount of PCR amplification product reference is an amount of PCR amplification product for the normal cell determined by a method as same as that of the RNA modification enzyme—deficient or the RNA demodification enzyme—deficient cells, or the RNA modification enzyme—low expressed or the RNA demodification enzyme—low expressed cells;

wherein the target site is a single gene-single site;

preferably, when the threshold cycle of PCR is less than the threshold cycle of PCR reference, it is determined that the chemical modification is performed by the RNA modification enzyme or the RNA demodification enzyme at the RNA target site,

alternatively, preferably, when the amount of PCR amplification product is more than the amount of PCR amplification product reference, it is determined that the chemical modification is performed by the RNA modification enzyme or the RNA demodification enzyme at the RNA target site.

In some embodiments of the present application, the RNA chemical modification is selected from the group consisting of m⁶A modification, m¹A modification, pseudouridine modification and 2′-O-methylation modification, preferably m⁶A modification; the RNA chemical modification enzyme includes m⁶A modification enzyme; preferably, the m⁶A modification enzyme is a methyltransferase complex or METTL16; the methyltransferase complex is selected from the group consisting of: METTL3, METTL14, WTAP, KIAA1429 (also known as VIRMA or VIRILIZER), HAKAI, ZC3H13, RBM15 and RBM15B, or combination thereof; the RNA demodification enzyme is FTO or ALKBH5.

The present application also provides a method for quantifying a RNA modification rate in transcripts, comprising:

(1) obtaining an RNA sample, and selecting a target RNA segment containing an RNA target site X in the RNA sample;

(2) determining an amount of the target RNA segment in the RNA sample, comprising:

(2a) randomly selecting an RNA non-target site N in the target RNA segment, preferably, the RNA non-target site N is located from 6^{th} nt of the upstream sequence of the RNA target site X to 2^{nd} nt of the downstream sequence of the RNA target site X; designing an up probe Pn1 and a down probe Pn2 for an upstream sequence and a downstream sequence of the RNA non-target site N, respectively, elongating the down probe Pn2 through a DNA polymerase to obtain an elongated down probe Pn2, and ligating the up probe Pn1 and the elongated down probe Pn2 through a ligase to obtain a SELECT product; performing PCR amplification of the SELECT product, and determining a threshold cycle N of PCR;

(2b) gradient diluting a reference sequence to a series of concentrations, obtaining a threshold cycle Nn of PCR corresponding to each concentration by the method of step (2a), and determining a standard curve 1 according to the concentrations and the threshold cycle Nn of PCR; preferably, the series of concentrations are between 0.1 fmol and 3 fmol, preferably between 0.2 fmol and 2.8 fmol, and more preferably between 0.2 fmol and 2.4 fmol;

wherein the reference sequence is a first reference sequence, a second reference sequence, or a mixture of the first reference sequence and the second reference sequence in any ratio,

the reference sequence comprises at least a nucleotide sequence II, and the nucleotide sequence II comprises a nucleotide sequence sharing a same nucleotide sequence with a nucleotide sequence I in the target RNA segment, wherein when the site N is located upstream of the site X, the nucleotide sequence I is a nucleotide sequence from a nucleotide that is complementary paired with a nucleotide of 3′-terminal of the up probe Pn1 of the site N to a nucleotide that is complementary paired with a nucleotide of 5′-terminal of the down probe Px2 of the site X in the target RNA segment, when the site N is located downstream of the site X, the nucleotide sequence I is a nucleotide sequence from a nucleotide that is complementary paired with a nucleotide of 3′-terminal of the up probe Px1 of the site X to a nucleotide that is complementary paired with a nucleotide of 5′-terminal of the down probe Pn2 at the site N in the target RNA segment,

and no target modification is present in an RNA target site X1 of the first reference sequence corresponding to the RNA target site X of the target RNA segment, and target chemical modification is present in an RNA target site X2 of the second reference sequence corresponding to the RNA target site of X of the target RNA segment;

preferably, a length of the reference sequence is at least 40 nt;

(2c) comparing the threshold cycle N of PCR with the standard curve 1, and determining the amount of the target RNA segment in the RNA sample;

(3) mixing the first reference sequence and the second reference sequence in a series of molarity ratios to obtain a series of mixtures, and applying the above (2) SELECT step and (3) PCR amplification to the mixtures to obtain a threshold cycle A1 of PCR or an amount A2 of PCR amplification product, determining a standard curve 2 according to the molarity ratios and the threshold cycle A1 of PCR or according to the molarity ratios and the amount A2 of PCR amplification product; preferably, mixing the RNA sample and the first reference sequence or the second reference sequence in the molarity ratios of 10:0, 8:2, 6:4, 4:6, 2:8 and 0:1;

(4) applying the above (2) SELECT step and (3) PCR amplification to the sample RNA to obtain a threshold cycle B1 of PCR or an amount B2 of PCR amplification product; and

(5) comparing the threshold cycle B1 of PCR or the amount B2 of PCR amplification product with the standard curve 2, to quantify the modification rate of the RNA target site X in the RNA sample.

In some embodiments of the present application, the RNA sample is total RNA, mRNA, rRNA, or lncRNA extracted from cells.

The present application provides a single-base elongation- and ligation-based PCR amplification method, which is used for detecting the chemical modifications in RNA at single-gene single-base resolution. The theory of the method is to exploit the ability of chemical modifications in RNA, such as m⁶A mark to hinder (i) the single-base elongation activity of DNA polymerases and (2) the nick ligation efficiency of ligases, and employs qPCR-based detection. The method is termed “SELECT”. In one preferred embodiment of the present application, two synthetic DNA oligos with PCR adapters (named the up probe and down probe) complementarily anneal to RNA but leave a nucleotide gap opposite to an m⁶A site. The chemical modifications, such as m⁶A modifications present in the RNA template selectively hinder Bst DNA polymerase mediated single-base elongation of the up probe. Importantly, although this first of two selection steps is not 100% efficient (a small number of elongation products will still be formed from a given modified site in an RNA template), the second nick ligation step filters these out. That is, any chemical modifications, such as m⁶A marks, in the RNA template serve to selectively prohibit nick ligation activity of ligase between the up probe and down probe. Thus, after two-round selection of chemical modifications, such as m⁶A marks, the amount of final ligation products formed from chemical modification, such as m⁶A-containing RNA templates is dramatically reduced compared to products formed from unmodified RNA templates, thus enabling simple qPCR-based quantification of chemical modification, such as m⁶A-modified versus unmodified target templates. FIG. 1 shows a schematic diagram of SELECT method for m⁶A detection.

The method of the present application can identify the chemical modification, such as m⁶A site in many types of RNA, such as rRNA, lncRNA, mRNA at single-base resolution precisely and efficiently; it can also quantify the modification fraction in RNA transcripts precisely; and can be used to identify a specific target site of various chemical modification enzyme, such as m⁶A modification enzyme. The method has a high sensitivity, which can be used in the detection of low-abundance RNA or ultralow-abundance RNA, and it is environment-friendly without using radioactive label.

### DESCRIPTION OF THE DRAWINGS

In order to illustrate the examples of the present application and the technical solutions of the prior arts more clearly, the drawings used in the examples and the prior arts are briefly described below. Obviously, the drawings in the following description are only some examples of the present application. For those ordinary skilled in the art, other drawings can be also obtained according to these drawings without any creative work.

FIG. 1 shows a schematic diagram of SELECT method for m⁶A detection. m⁶A in RNA is selected twice in a one-tube reaction: in the first selection step, an m⁶A mark hinders the ability of DNA polymerase to elongate the target sequence by preventing the addition of a thymidine on the down probe opposite to the m⁶A site; in the second selection step, m⁶A marks that are present in the RNA template selectively prohibit DNA-ligase-catalyzed nick ligation between the up probe and the down probe; the final elongated and ligated products are then quantified by qPCR.

FIG. 2 shows the evaluation on site N selection. (a) the bar plot of the threshold cycle (C_{T}) of qPCR, showing SELECT results for detecting X, X−1, X−2, X−4, X−6, X+1 and X+2 sites in Oligo1-m⁶A and Oligo1-A; (b) the bar plot of the threshold cycle (C_{T}) of qPCR, showing SELECT results for detecting X, X−1, X−2, X−4, X−6 sites in Oligo2-m⁶A and Oligo2-A; 1 fmol RNA is used in this assay; error bars indicate mean±s.d for 2 biological replicates x 2 technical replicates.

FIG. 3 shows the optimized SELECT results for detecting m⁶A in the model oligonucleotides. (a) the real-time fluorescence amplification curves and bar plot of the threshold cycle (C_{T}) of qPCR, showing SELECT results for detecting the site X and site N (for input control) in Oligo1-m⁶A versus Oligo1-A; (b) the real-time fluorescence amplification curves and bar plot of the threshold cycle (C_{T}) of qPCR, showing SELECT results for detecting the site X and site N (for input control) in Oligo2-m⁶A versus Oligo2-A; error bars indicate mean±s.d for 3 biological replicates x 2 technical replicates; Rn is the raw fluorescence for the associated well normalized to the fluorescence of the passive reference dye (ROX).

FIG. 4 shows the results of the combination of SELECT method with PCR and TBE-PAGE for detecting the model of Oligo1-m⁶A versus Oligo1-A.

FIG. 5 shows the results of verifying the selectivity of SELECT method by mixing Oligo1-m⁶A and Oligo1-A in different ratios. (a) real-time fluorescence amplification curves, showing SELECT results for detecting the mixture Oligo1-m6A with Oligo1-A with known m⁶A ratios; (b) the linear relationship between the relative products of SELECT (2C_{T} values normalized to 2% of the 2C_{T} value of 100% m⁶A) and m⁶A fraction.

FIG. 6 shows the bar plot of the threshold cycle (C_{T}) of qPCR (left y axis) and the line plot of different C_{T} (ΔC_{T}) (right y axis), showing the results of performance test of 7 ligases used in the SELECT method: SplintR ligase (a), T3 DNA ligase (b), T4 RNA ligase 2 (c), T4 DNA ligase (d), T7 DNA ligase (e), 9° N™ DNA ligase (f) and Taq DNA ligase (g). Error bars indicate mean±s.d for 2 biological replicates x 2 technical replicates.

FIG. 7 shows the bar plot of the C_{T} of qPCR (left y axis) and the line plot of different C_{T} (ΔC_{T}) (right y axis), showing the optimization of following reaction conditions: temperature (a), dTTP concentration (b), Bst 2.0 DNA polymerase amount (c) and SplintR ligase amount (d) in SELECT for detecting site X in Oligo1-m⁶A and Oligo1-A. Error bars indicate mean±s.d for 2 biological replicates x 2 technical replicates.

FIG. 8 shows the effects of dTTP and dNTP on site X and site N in Oligo1-m⁶A and Oligo1-A by the SELECT detection method. Error bars indicate mean±s.d for 3 biological replicates x 2 technical replicates.

FIG. 9 shows the amplification efficiency of qPCR primers used in SELECT method. SELECT method detects the DNA fragments produced by Oligo1, in TA clone pGEM-T vector. (a) Oligo1 qPCR amplicon sequence confirmed by Sanger sequencing; (b) linear relationship between C_{T} and recombinant plasmid concentration lg. The amplification efficiency of the designed qPCR primer is 97.2% calculated by the slope of −3.39. Error bars indicate mean±s.d for 2 biological replicates x 3 technical replicates.

FIG. 10 shows the results of more down probes used in SELECT method, in which the first nucleotide of the 3′ terminal of the down probe is complementary paired with the nucleotide located at a site with a distance of 2 nt (a), 3 nt (b) and 4 nt (c) from the RNA target site X at the downstream sequence of the RNA target site X.

FIG. 11 shows the results of the combination of SELECT with an FTO-assisted demethylation step for detecting m⁶A sites in total RNA or polyA-RNA. (a) the FTO-assisted SELECT m⁶A detection method; (b) coomassie blue staining of recombinant FTO protein purified from E. coli; (c) UPLC-MS/MS detection for the content of m⁶A in RNA, the m⁶A demethylation activity of FTO in total RNA or polyA-RNA isolated from HeLa or HEK293T cells, EDTA chelates the cofactor Fe²⁺ and inactivates FTO.

FIG. 12 shows real-time fluorescence amplification curves and bar plot of the threshold cycle (C_{T}) of qPCR, showing SELECT results for detecting m⁶A4190 and A4194 sites (for input control) in A2511 of 28S rRNA (30 ng) of HeLa cells.

FIG. 13 shows real-time fluorescence amplification curves and bar plot of the threshold cycle (C_{T}) of qPCR, showing SELECT results for detecting m⁶A2515 and m⁶A2577, m⁶A2611, A2511 and A2624 (for input control) in lncRNA MALAT1 (10 ng) of HeLa cells.

FIG. 14 shows real-time fluorescence amplification curves and bar plot of the threshold cycle (C_{T}) of qPCR, showing SELECT results for detecting m⁶A1211 and A1207 (for input control) in mRNA H1F0 (1 μg) of HEK293 cells.

FIG. 15 shows the putative m⁶A site in mRNA H1F0 of HEK293 cells in several reported m⁶A sequencing data.

FIG. 16 shows polyacrylamide gel electrophoresis (PAGE) results of PCR amplification of the elongated and ligated products, by using FTO-assisted SELECT for detecting m⁶A4190 and m⁶A4194 (for input control) sites in 28S rRNA and m⁶A2577 and A2614 (for input control) in lncRNA MALAT1. For m⁶A4190, A4194, m⁶A2577 and A2614 sites, the length of PCR product is 79 bp, 79 bp, 100 bp and 101 bp, respectively, and the cycle of PCR is 22, 21, 29 and 25, respectively.

FIG. 17 shows the C_{T} bar plot of the FTO-assisted SELECT results for detecting the m⁶A2577 site (a) and A2614 site (b) in lncRNA MALAT1 by using different amounts of polyA-RNA; error bars indicate mean±s.d for 2 biological replicates x 3 technical replicates.

FIG. 18 shows SELECT results for quantifying the m⁶A fraction in the transcripts.

FIG. 19 shows SELECT results for the identification of the biological target site of the m⁶A modification enzyme METTL3. (a) SELECT in combination with genetics methods used for the identification of the biological target site of m⁶A modification enzymes; (b) Western blotting shows that the METTL3 protein level is reduced in METTL3^{+/-} HeLa heterozygous cells; (c) UPLC-MS/MS shows the total m⁶A levels in control cells and METTL3^{+/-} HeLa heterozygous cells; (d) real-time fluorescence amplification curves and bar plot of the threshold cycle (C_{T}) of qPCR, showing SELECT results for detecting m⁶A2515 and A2511 (for input control) in lncRNA MALAT1 in control versus METTL3^{+/-} cells, establishing that the 2515 site of MALAT1 is a biological target site of METTL3. Error bars indicate mean±s.d for 2 biological replicates×3 technical replicates.

FIG. 20 shows SELECT results for the identification of the biological target site of the m⁶A modification enzyme METTL3. (a) linear relationship between C_{T} of qPCR of MALAT1 and relative concentration 1 g of reverse transcription mixture, the amplification efficiency of the designed qPCR primer of MALAT1 is 102.7% calculated by the slope of −3.26; (b) the real-time fluorescence amplification curves of the MALAT1 segment in the control and METTL3^{+/-} samples, and the C_{T} is shown in the table. The amount of total RNA is measured by Qubit, and the amount of MALAT1 in the total RNA from the control and METTL3^{+/-} samples is quantified by qPCR. The amount of MALAT1 in METTL3^{+/-} is 1.526 times as much as in the control, calculated by 2ΔC^{T} method. 2 μg total RNA from METTL3^{+/-} cells and 3.05 μg total RNA from control cells are used in this assay; error bars indicate mean±s.d for 2 biological replicates x 3 technical replicates.

FIG. 21 shows the real-time fluorescence amplification curves and bar plots, showing SELECT results for detecting other types of RNA modifications. It shows SELECT results for detecting the site X and site N of Oligo4-m¹A versus Oligo4-A (a), Oligo1-Am versus Oligo1-A (b), and Oligo5-Ψ versus Oligo5-U (c). 1 fmol RNA is used in this assay. Error bars indicate mean±s.d for 2 biological replicates x 3 technical replicates.

### DETAILED DESCRIPTION OF THE INVENTION

In order to illustrate the objects, technical solutions, and advantages of the present application more clearly, the present application is further described in detail with reference to the drawings and examples. Unless otherwise specified, the reagents and experimental materials used in the examples are all conventional commercially available reagents and experimental materials, and the methods used in the examples are well known and conventional methods to those skilled in the art.

### Experimental Methods

1. Cell Culture and RNA Extraction

HeLa cells, HEK293T cells, and METTL3^{+/-} HeLa heterozygous cells produced by CRISPR/cas9 were cultured in DMEM medium (purchased from Corning) containing 10% FBS (purchased from Gibco) and 1% penicillin-streptomycin (purchased from Corning) at 37° C. and 5% CO₂. According to the manufacturer's instructions, total RNA was extracted with TRIzol reagent (purchased from ThermoFisher Scientific). Two rounds of polyA selection were carried out from total RNA with Dynabeads Oligo (dT)₂₅ (purchased from ThermoFisher Scientific, item number 61002) according to the manufacturer's instructions to isolate PolyA-RNA.

2. Western Blotting

The protein levels of METTL3 in control cells and METTL3^{+/-} HeLa heterozygous cells were detected by Western blotting. The METTL3^{+/-} HeLa heterozygous cells were obtained by CRISPR/Cas9 knockout, and the control cells are HeLa cells obtained through CRISPR/Cas9 by using non-targeted sgRNA, the METTL3 gene in the control cells was not knockout as described above. Briefly, the control cells and METTL3^{+/-} cells were collected, mixed with 2×SDS loading buffer (100 mM Tris-HCl, pH 6.8, 1% SDS, 20% glycerol, 25% β-mercaptoethanol, 0.05% bromophenol blue) and incubated at 95° C. for 15 minutes. After centrifugation at 12,000 rpm, the samples were separated by SDS-PAGE and transferred from the gel to the PVDF membrane. Antibody staining was performed with METTL3 antibody (purchased from Cell Signaling Technology) and ACTIN antibody (purchased from CWBIO). Finally, the film was imaged by the Tanon 5500 chemiluminescence imaging system.

3. Select Method

Total RNA, polyA-RNA or synthetic RNA oligonucleotides were mixed with 40 nM up probe, 40 nM down probe and 5 μM dTTP (or dNTP) in 17 μl 1× CutSmart buffer (50 mM potassium acetate, 20 mM Tris-acetic acid, 10 mM magnesium acetate, 100 μg/ml BSA, pH 7.9, at 25° C.). The probe and RNA were annealed by incubating the mixture under the following temperature gradient: 90° C., 1 minute; 80° C., 1 minute; 70° C., 1 minute; 60° C., 1 minute; 50° C., 1 minute, then 40° C., 6 minutes. Subsequently, a 3 μl mixture containing 0.01 U Bst 2.0 DNA polymerase, 0.5 U SplintR ligase and 10 nmol ATP was added to the mixture to obtain a final reaction mixture with a volume of 20 μl. The final reaction mixture was incubated at 40° C. for 20 minutes, denatured at 80° C. for 20 minutes and kept at 4° C. to obtain the SELECT product.

4. qPCR

The SELECT product obtained in step 3 was subjected to a real-time quantitative PCR (qPCR) reaction in Applied Biosystems ViiA™ 7 real-time PCR system (Applied Biosystems, USA). The 20 μl qPCR reaction system was consisted of 2×Hieff qPCR SYBR Green Master Mix (purchased from Yeasen), 200 nM qPCR upstream primer (qPCRF), 200 nM qPCR downstream primer (qPCRR), 2 μl of the above SELECT product and the balance of ddH₂O. qPCR was run under the following conditions: 95° C., 5 minutes; (95° C., 10 s, 60° C., 35 s)×40 cycles; 95° C., 15s; 60° C., 1 minute; 95° C., 15s (the fluorescence was collected at a heating rate of 0.05° C./s); and kept at 4° C. The data was analyzed by QuantStudio™ Real-Time PCR software v1.3.

5. TBE-PAGE Electrophoresis Analysis of PCR Products

Before qPCR, 2 μl of SELECT product was mixed with 2×Taq Plus Master Mix (purchased from Vazyme), 400 nM qPCR upstream primer, 400 nM qPCR downstream primer to obtain a total volume of 25 μl of the mixture. Then, PCR of the site X (29 cycles) and site N (26 cycles) was carried out. 10 μl PCR products were subjected to electrophoresis on a 12% non-denaturing TBE-PAGE gel with 0.5% TBE buffer in an ice bath. TBE-PAGE gel was stained with YeaRed nucleic acid gel stain (purchased from Yeasen), and photographed with Tanon 1600 gel imaging system (Tanon).

6. Ligation and qPCR Based on Different Ligases

80 thiol of synthetic RNA oligonucleotide was mixed with 40 nM T upstream primer (SEQ ID NO. 6) and 40 nM downstream primer (SEQ ID NO. 7) in 18 μl 1× reaction buffer. It should be noted that, compared with the primers used in SELECT, the T upstream primer was introduced one more base T at the 3′ terminal. The base T was necessary to be artificially introduced at the 3′ terminal because no DNA polymerase was used for reverse transcription in the method to synthesize T opposite to m⁶A or A. 1× CutSmart buffer (50 mM potassium acetate, 20 mM Tris-acetic acid, 10 mM magnesium acetate, 100 μg/ml BSA, pH 7.9, at 25° C.) was used to detect SplintR ligase, T4 DNA ligase and T4 RNA ligase 2 (dsRNA ligase).

1×T3 DNA ligase reaction buffer (66 mM Tris-HCl, 10 mM MgCl₂, 1 mM ATP, 1 mM DTT, 7.5% PEG 6000, pH 7.6, at 25° C.) was used to detect T3 DNA ligase and T7 DNA ligase.

1×9° N DNA ligase reaction buffer (10 mM Tris-HCl, 600 μM ATP, 2.5 mM MgCl₂, 2.5 mM DTT, 0.1% Triton X-100, pH 7.5, at 25° C.) was used to detect 9° N DNA ligase.

1×Taq DNA ligase reaction buffer (20 mM Tris-HCl, 25 mM potassium acetate, 10 mM magnesium acetate, 10 mM DTT, 1 mM NAD, 0.1% Triton X-100, pH 7.6, at 25° C.) was used to detect Taq DNA ligase.

The probe and RNA were annealed by incubating the mixture under the following temperature gradient: 90° C., 1 minute; 80° C., 1 minute; 70° C., 1 minute; 60° C., 1 minute; 50° C., 1 minute; then 40° C., 6 minutes. 2₁1.1 of a mixture containing 10 nmol ATP and ligase with the specified concentration was added (only added in the detection of SplintR ligase, T4 DNA ligase and T4 RNA ligase 2) to the above annealed mixture. The final reaction mixture was reacted at 37° C. for 20 minutes, then denatured at 95° C. for 5 minutes, and kept at 4° C. Subsequently, qPCR was carried out in the same manner as in step 3.

7. Clone, expression and purification of recombinant FTO protein

The truncated human FTO cDNA (ΔN31) was subcloned into the pET28a vector. The plasmid was transformed into BL21-Gold (DE3) E. coli competent cells. The expression and purification of the FTO protein were performed according to procedures well known to those skilled in the art (for example, see G. Jia, et al., Nat. Chem. Biol. 2011, 7, pages 885-887). The purified FTO protein was identified by 12% SDS-PAGE electrophoresis.

8. FTO-Mediated Demethylation of m⁶A

The total RNA or polyA-RNA was treated with FTO protein according to methods well known to those skilled in the art (see, for example, G. Jia, et al., Nat. Chem. Biol. 2011, 7, pages 885-887). For the experimental group: 40 μg total RNA or 2 μg polyA-RNA was mixed with FTO, 50 mM HEPES (pH 7.0), 2 mM L-ascorbic acid, 300 μM α-ketoglutarate (α-KG), 283 μM (NH₄)₂Fe(SO₄)₂.6H₂O and 0.2 U/μl RiboLock RNase inhibitor (purchased from ThermoFisher Scientific), and reacted at 37° C. for 30 minutes. The reaction was quenched by adding 20 mM EDTA. For the control group: 20 mM EDTA should be added before the demethylation reaction. The RNA was recovered by phenol-chloroform extraction and ethanol precipitation, and then detected by the SELECT method.

9. Quantification of m⁶A by UPLC-MS/MS

200 ng RNA was digested with 1 U nuclease P1 (purchased from Wako) in 10 mM ammonium acetate buffer at 42° C. for 2 hours, and then incubated with 1 U rSAP (purchased from NEB) in 100 mM MES (pH6.5) at 37° C. for 4 hours. The digested sample was centrifuged at 15,000 rpm for 30 minutes, and 5 μl of the solution was injected into UPLC-MS/MS. The nucleotides were separated by ZORBAX SB-Aq column (Agilent) in UPLC (SHIMADZU), and detected by Triple Quad™ 5500 (AB SCIEX). The nucleotides were quantified based on the m/z transition of the parent ions and daughter ions: for A, m/z is 268.0 to 136.0, for m⁶A, m/z is 282.0 to 150.1. The commercially available nucleotides were used to plot a standard curve, and the ratio of m⁶A/A was calculated precisely according to the standard curve.

In the context, the term “threshold cycle (C_{T})”, also known as the threshold cycle value, refers to the number of amplification cycles when the fluorescence signal of the amplification product reaches the set fluorescence threshold during the qPCR amplification process.

In the context, the term “upstream” refers to the position and/or direction away from the transcription or translation initiation site in the DNA sequence or messenger ribonucleic acid (mRNA), that is, the position close to the 5′ terminal or the direction toward the 5′ terminal. The term “downstream” refers to the position and/or direction away from the transcription or translation initiation site in the DNA sequence or messenger ribonucleic acid (mRNA), that is, the position close to the 3′ terminal or the direction toward the 3′ terminal.

In the context, the term “a nucleotide located at a site with a distance of 1 nt from the RNA target site X at the upstream sequence of the RNA target site X” refers to a nucleotide at the position adjacent to the RNA target site X at the upstream sequence of the RNA target site X. For example, if the RNA target site X is defined as the 0 th position, then the nucleotide located at a site with a distance of 1 nt from the RNA target site X at the upstream sequence of the RNA target site X is at −1 position, and the nucleotide located at a site with a distance of 1 nt from the RNA target site X at the downstream sequence of the RNA target site X is at +1 position

In the context, RNA modification enzyme refers to an enzyme capable of chemically modifying the nucleotides in RNA. For example: m⁶A modification enzyme can convert A into m⁶A, m⁶A modification enzyme includes, for example, (1) methyltransferase complex and (2) METTL16. The methyltransferase complex is selected from the group consisting of METTL3, METTL14, WTAP, KIAA1429 (also known as VIRMA or VIRILIZER), HAKAI, ZC3H13, RBM15 and RBM15B, or combination thereof. The enzymes that form m¹A modification, pseudouridine modification and 2′-O-methylation modification in RNA also belong to RNA modification enzymes.

In the context, RNA demodification enzyme refers to an enzyme that removes chemical modifications on nucleotides in RNA and converts the modified nucleotides into ordinary A, U, C or G. FTO and ALKBH5 are demodification enzyme of m⁶A. The m⁶A modification and the m¹A modification are converted to A under the action of the demodification enzyme. The pseudouridine modification is converted to U under the action of the demodification enzyme.

TABLE 1
Model RNA oligonucleotides used in
the present application

| Name | Sequence (5′->3′) | Features |
|---|---|---|
| Oligo1 | rArUrGrGrGrCrCrGrUrUr | X represents A, |
| | CrArUrCrUrGrCrUrArArA | m6A or Am, |
| | rA [symbol] rGrCr | wherein Am |
| | UrUrUrUrGrGrGrGrCrUr | represents 2′-O- |
| | UrGrU | methyl adenosine |
| Oligo2 | rArUrGrGrGrCrCrGrUrU | X represents |
| | rCrAtUrCrUrGrCrUrArA | A or m⁶A |
| | rArA [symbol] rGrC | |
| | rUrUrUrUrGrGrGrGrCrU | |
| | rUrGrU | |
| Oligo3 | rArGrUrArGrCrUrUrArG | X represents |
| | rUrUrUrGrArArArArArU | A or m⁶A |
| | rGrUrGrArA | |
| | [symbol] rUrUr | |
| | CrGrUrArArCrGrGrAr | |
| | ArGrUrArArUrUrC | |
| Oligo4 | rUrGrGrGrGrUrCrUrC | X represents A, |
| | rCrCrCrGrCrGrCrArG | m⁶A (N1- |
| | rGrUrUrCrG [symbol] rArUrC | methyl adenosine) |
| | rCrCrGrCrCrGrArGrU | |
| | rArCrGrU | |
| | rCrA | |
| Oligo5 | rGrGrGrGrArArGrArG | X represents |
| | rCrArArCrArArArGrC | U or Ψ |
| | rArArGrCrArArGrArC | |
| | rGrArCrArArGrGrArA | |
| | rGrCrArArArArCrArA | |
| | rCrArCrGrCrCrArGrA | |
| | rCrArCrGrGrGrArArG | |
| | rArG [symbol] rCrArGrArCrG | |
| | rArCrCrArCrArCrGrA | |
| | rArGrArArCrCrArCrA | |
| | rCrArGrArGrCrArArG | |
| | rGrArArArCrArCrCrA | |
| | rArCrArCrCrArCrCrA | |
| | rCrCrGrCrArGrArGrA | |
| | rGrArGrArArArGtGrG | |
| | rArCrArGrGrGrArCrA | |
| | rCrCrArArGrCrArGrG | |
| | rCrArCrArGrArArCrA | |
| | rArG | |
| Note: | | |

1. The lowercase letter r to the left of bases A, U, C, and G indicates (hat the nucleotide is a ribonucleotide;
2. The underlined part represents the classical conservative motif of m⁶A.

TABLE 2
Primers used in qPCR in step 6 of
the experimental method

| Name | Sequence (5′->3′) |
|---|---|
| Oligo1-X-T- | tagccagtaccgtagtgcgtg |
| upstream | AGCCCCAAAAGCAGT (SEQ |
| primer | ID NO. 6) |
| Oligo1-X- | 5phos/CCTTTTAGCAGATGAA |
| downstream | CGGCcagaggctgagtcgc |
| primer | tgcat (SEQ ID NO. 7) |

Note:
5phos represents 5′phosphorylation.

TABLE 3
Probes used in the SELECT method of
the present application

| Name | Sequence (5′->3′) |
|---|---|
| Oligo3-X- | Tagccagtaccgtagtgcg |
| downstream | tgAGCCCCAAAAGCAG |
| probe/ | (SEQ ID NO. 8) |
| Oligo2-X- | |
| downstream | |
| probe | |
| Oligo1-X- | 5phos/CCTTTTAGCAGA |
| upstream | TGAACGGCcagag |
| probe | gctgagtcgctgcat |
| | (SEQ ID NO. 9) |
| Oligo2-X- | 5phos/TCTTTTAGCAGA |
| upstream | TGAACGGCcagag |
| probe | gctgagtcgctgcat |
| | (SEQ ID NO. 10) |
| Oligo1-X − 1- | Tagccagtaccgtagtgc |
| downstream | gtgAGCCCCAAAAGCAG |
| probe | T (SEQ ID NO. 31) |
| Oligo2-X − 1- | Tagccagtaccgtagtgc |
| downstream | gtgAGCCCCAAAAGCAG |
| probe | T (SEQ ID NO. 12 ) |
| Oligo1-X − 1- | 5phos/CTTTTAGCAGAT |
| upstream | GAACGGCcagaggc |
| probe/ | tgagtcgctgcat |
| Oligo2-X − 1- | (SEQ ID NO. 13) |
| upstream | |
| probe | |
| Oligo1-X − 2- | tagccagtaccgtagtgc |
| downstream | gtgAGCCCCAAAAGCAG |
| probe | TC (SEQ ID NO. 14) |
| Oligo2-X − 2- | tagccagtaccgtagtgc |
| downstream | gtgAGCCCCAAAAGCAG |
| probe | TT (SEQ ID NO. 15) |
| Oligol-X − 2- | 5phos/TTTTAGGAGATG |
| upstream | AACGGCcagaggctg |
| probe/ | agtcgctgcat |
| Oligo2-X − 2- | (SEQ ID NO. 16) |
| upstream | |
| probe | |
| Oligo1-X − 4- | tagccagtaccgtagtg |
| downstream | cgtgAGCCCCAAAAGCAG |
| probe | TCCT |
| | (SEQ ID NO. 17) |
| Oligo2-X − 4- | tagccagtaccgtagtg |
| downstream | cgtgAGCCCCAAAAGCAG |
| probe | TTCT |
| | (SEQ ID NO. 18) |
| Oligo1-X − 4- | 5phos/TTAGCAGATGAA |
| upstream | CGGCcagaggagagt |
| probe/ | cgctgcat |
| Oligo2-X − 4- | (SEQ ID NO. 19) |
| upstream | |
| probe | |
| Oligo3-X − 6- | tagccagtaccgtagtgcg |
| downstream | tgAGCCCCAAAAGCAG |
| probe | TCCTTT |
| | (SEQ ID NO. 20) |
| Oligo2-X − 6- | tagccagtaccgtagtgcg |
| downstream | tgAGCCCCAAAAGCAG |
| probe | TTCTTT (SEQ ID NO. 23) |
| Oligo1-X − 6- | 5phos/AGCAGATGAAC |
| upstream | GGCcagaggctgagtcg |
| probe/ | ctgcat (SEQ ID NO. 22) |
| Oligo2-X − 6- | |
| upstream | |
| probe | |
| Oligo1-X + 1- | tagccagtaccgtagtgc |
| downstream | gtgAGCCCCAAAAGCA |
| probe | (SEQ ID NO. 23) |
| Oligo1-X + 1- | 5phos/TCCTTTTAGCAG |
| upstream | ATGAACGGCcaga |
| probe | ggctgagtcgctgcat |
| | (SEQ ID NO. 24) |
| Oligo1-X + 2- | tagccagtaccgtagtgc |
| downstream | gtgACAAGCCCCAAAAG |
| probe | C (SEQ ID NO. 25) |
| Oligo1-X + 2- | 5phos/GTCCTTTTAG |
| downstream | CAGATGAACGGCcag |
| | aggctgagtcgctgcat |
| | (SEQ ID NO. 26) |
| Oligo4-X- | tagccagtaccgtagtgc |
| downstream | gtgTGACGTAGTCGGCA |
| probe | GGAT |
| | (SEQ ID NO. 27) |
| Oligo4-X- | 5phos/CGAACCTGCGCG |
| upstream | GGGcagaggctgagtcg |
| probe | ctgcat |
| | (SEQ ID NO. 28) |
| Oligo4-X − 7- | tagccagtaccgtagtgc |
| downstream | gtgGTCGGCAGGATTCG |
| probe | AACC |
| | (SEQ ID NO. 29) |
| Oligo4-X − 7- | 5phos/GCGCGGGGAGAC |
| upstream | CCCcagaggctgagtc |
| probe | gctgcat |
| | (SEQ ID NO. 30) |
| Oligo5-X- | tagccagtaccgtagtgc |
| downstream | gtgCTTCGTGTGGTCGTC |
| probe | TG |
| | (SEQ ID NO. 31) |
| Oligo5-X- | 5phos/CTCTTCCCGTGT |
| upstream | GTGGcagaggctgagtc |
| probe | gctgcat |
| | (SEQ ID NO. 32) |
| Oligo4-X + 4- | tagccagtaccgtagtgc |
| downstream | gtgTGGTTCTTCGTGTGG |
| | TCG |
| | (SEQ ID NO. 33) |
| Oligo4-X + 4- | 5phos/CTGACTCTTCCC |
| upstream | GTGTGcagaggctgag |
| probe | tcgctgcat |
| | (SEQ ID NO. 34) |
| 28S_m6A4190_ | Tagccagtaccgtagtgc |
| downstream | glgCGCCTTAGGACACC |
| probe | TGCG |
| | (SEQ ID NO. 35) |
| 28S_m6A4190_ | 5phos/TACCGTTTGACA |
| upstream | GGTGTAcagaggctg |
| probe | agtcgctgcat |
| | (SEQ ID NO. 36) |
| 28S_A4194- | tagccagtaccgtagtgc |
| downstream | gtgAGCTCGCCTTAGGA |
| probe | CACC |
| | (SEQ ID NO. 37) |
| 28S_A4194- | 5pbos/GCGT7ACCGITT |
| upstream | GACAGGTcagaggc |
| probe | tga |
| | gtcgctgcat |
| | (SEQ ID NO. 38) |
| MALAT1_m⁶A2515_ | tagccagtaccgtagtgc |
| downstream | gtgAATTACTTCCGTTAC |
| probe | GAAAG |
| | (SEQ ID NO. 39) |
| MALAT1_m⁶A2515_ | 5phos/CCTTCACATTTT |
| upstream | TCAAACTAAGCTACTca |
| probe | gaggctgagtcgctgcat |
| | (SEQ ID NO. 40) |
| MALAT1_m⁶A2577_ | tagccagtaccgtagtgc |
| downstream | gtgGGATTTAAAAAATA |
| probe | ATCTTAACTCAAAG |
| | (SEQ ID NO. 41) |
| MALAT1_m⁶A2577_ | 5phos/CCAATGCAAAAA |
| upstream | CATTAAGTcagaggctg |
| probe | agtcgctgcat |
| | (SEQ ID NO. 42) |
| MALAT1_A2511_ | tagccagtaccgtagtgc |
| downstream | gtgAATTACTTCCGTTAC |
| probe | GAAAGTCCT |
| | (SEQ ID NO. 43) |
| MALAT1_A2511_ | 5phos/CACATTTTTCAA |
| upstream | ACTAAGCTACTcagagg |
| probe | ctgagtcgctgcat |
| | (SEQ ID NO. 44) |
| MALAT1_m⁶A2611- | tagccagtaccgtagtg |
| downstream probe | cgtgGTCAGCTGTCAAT |
| | TAATGC |
| | (SEQ ID NO. 45) |
| MALAT1_m⁶A2611- | 5phos/AGTCCTCAGGAT |
| upstream probe | TTAAAAAATAATCTTAAC |
| | cagaggctgagtcgctg |
| | cat |
| | (SEQ ID NO. 46) |
| H1F0-m6A1211- | Tagccagtaccgtagtgc |
| downstream probe | gtgCATTAGATTGGTTGT |
| | TGCTG |
| | (SEQ ID NO. 47) |
| H1F0-m6A1211- | 5phos/CCTTGCACAACT |
| upstream probe | GGTTAAcagaggctg |
| | agtcgctgcat |
| | (SEQ ID NO. 48) |
| H1F0-A1207- | tagccagtaccgtagtg |
| downstream | cgtgTGGTTGTTGCTGT |
| probe | CCT |
| | (SEQ ID NO. 49) |
| H1F0-A1207- | 5phos/GCACAACTGGT |
| npstream probe | TAAGGAAAcagaggct |
| | gagtcgctgcat |
| | (SEQ ID NO. 50) |

TABLE 4
Primers used for qPCR of SELECT products

| Name | Sequence (5′-> 3+40) |
|---|---|
| qPCRF | ATGCAGCGACTCAGCCTCTG |
| | (SEQ ID NO. 51) |
| qPCRR | TAGCCAGTACCGTAGTGCGTG |
| | (SEQ ID NO. 52) |
| MALAT1_gPCRF | GACGGAGGTTGAGATGAAGCT |
| | (SEQ ID NO. 53) |
| MALAT1_gPCRR | ATTalOGGCTCTGTAGTCCT |
| | (SEQ ID .NO. 54) |

### Example 1 SELECT Method in Combination with qPCR for Detecting m⁶A Modification in Model m⁶A RNA Oligonucleotide

Two kinds of model 42-mer RNA Oligos with an internal site X (X=m⁶A or A): Oligo1 (SEQ ID NO.1) and Oligo2 (SEQ ID NO.2) were subjected to SELECT method. According to whether there is a methylation modification at the site X, the model oligonucleotides were divided into 4 categories: Oligo1-m⁶A, Oligo1-A, Oligo2-m⁶A, and Oligo2-A.

(1) Controlling the Initial RNA Input Amounts

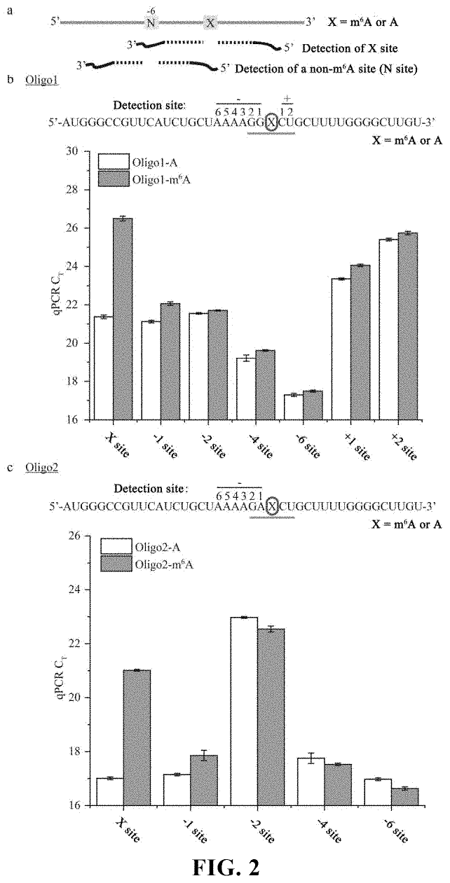

Given that the initial RNA input amounts directly affected the OCR amplification cycles, the inventors simultaneously detected a non-m⁶A modification site (also called site N) in model oligonucleotides to control the initial RNA input amounts (FIG. 2a). In theory, a same threshold cycle (C_{T}) of OCR will be detected by SELECT for an site N in both Oligo1-m⁶A and Oligo1-A, indicating that the initial RNA input amounts are equal; in the same way, a same threshold cycle of OCR will be detected by SELECT for an site N in both Oligo2-m⁶A and Oligo2-A.

The inventors performed SELECT at 6^{th} nt of the upstream sequence of site X to 2^{nd} nt of the downstream sequence of site X (X−6 to X+2) in order to determine site N. The results showed that any non-m⁶A modification site except the site of 1 bp upstream and downstream of m⁶A site (m⁶A±1) can be used as an site N for controlling the initial RNA input amounts (see FIGS. 2b and 2c). In this example, the X−6 site (i.e., at 6^{t11} nt of the upstream sequence of site X) was set as the site N in each model oligonucleotide for controlling the initial RNA input amounts.

(2) SELECT Method in Combination with qPCR for Detecting m⁶A Modification in Model m⁶A RNA Oligonucleotides

According to the SELECT method in step 3 of the above experimental methods, the Bst 2.0 DNA polymerase and SplintR ligase were reacted with Oligo1-m⁶A, Oligo1-A, Oligo2-m⁶A, and Oligo2-A, to obtain Oligo1-m⁶A, Oligo1-A, Oligo2-m⁶A, and Oligo2-A products of SELECT, respectively.

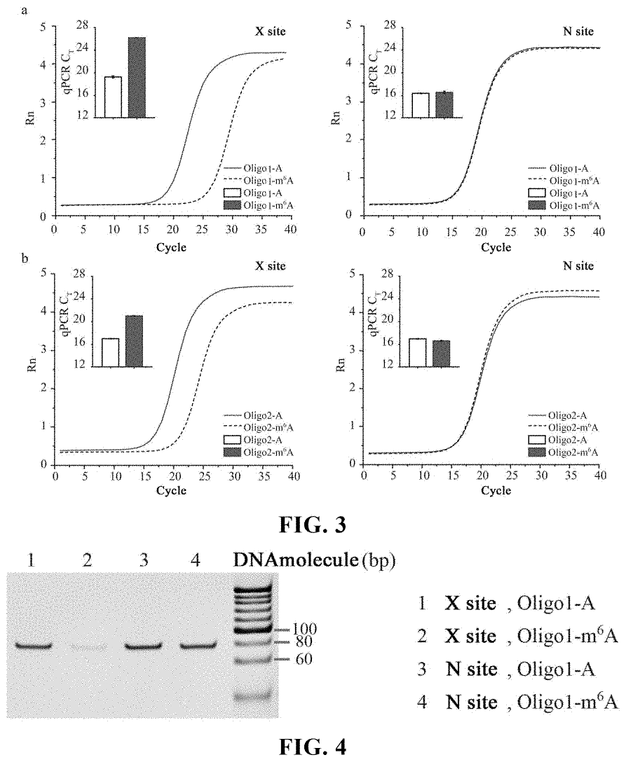

The SELECT products were subjected to qPCR in Applied Biosystems ViiA™7 real-time PCR system (Applied Biosystems, USA). The data was analyzed by QuantStudio™ Real-Time PCR software v1.3. FIGS. 3a and 3b showed SELECT results for detecting site X (FIG. 3a, left, and FIG. 3b, left) and site N (FIG. 3a, right, and FIG. 3b, right) in Oligo1-m⁶A, Oligo1-A, Oligo2-m⁶A, and Oligo2-A, respectively, in which results of site N were input control.

It can be seen that, when controlling the RNA input amounts to be same (i.e., C_{T}s of amplification of the site N for Oligo1-m⁶A versus Oligo1-A were same; C_{T}s of amplification of the site N for Oligo2-m⁶A versus Oligo2-A were same), the threshold cycle difference of amplification (ΔC_{T}) of the site X for Oligo1-m⁶A versus A-oligo was up to 7.6 cycles for Oligo1 containing a GGXCU sequence and 4 cycles for Oligo2 containing a GAXCU sequence (FIGS. 3a and 3b), demonstrating that the SELECT method of the present application can efficiently distinguish m⁶A-modified sites from unmodified sites.

### Example 2 SELECT Method in Combination with PCR and TBE-PAGE for Detecting m⁶A Modification in Model m⁶A RNA Oligonucleotide

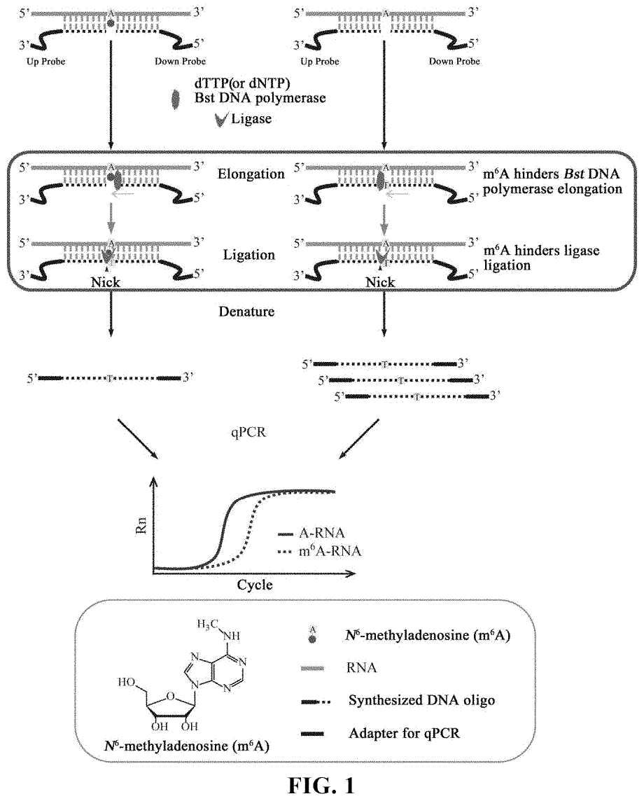

The SELECT products of Oligo1-m⁶A and Oligo1-A obtained in Example 1 were subjected to PCR by using experimental method 3 and then subjected to TBE-PAGE electrophoresis analysis. FIG. 1c showed the results of TBE-PAGE gel electrophoresis. It can be seen that, compared with the site N of Oligo1-m⁶A and Oligo1-A and the site X of Oligo1-A, almost no band of PCR product was observed for the site X of Oligo1-m⁶A. It can be seen that the SELECT method of the present application has significant selectivity for m⁶A compared to adenosine (A) without methylation modification (FIG. 4).

### Example 3 Verification of the Selectivity of SELECT Method

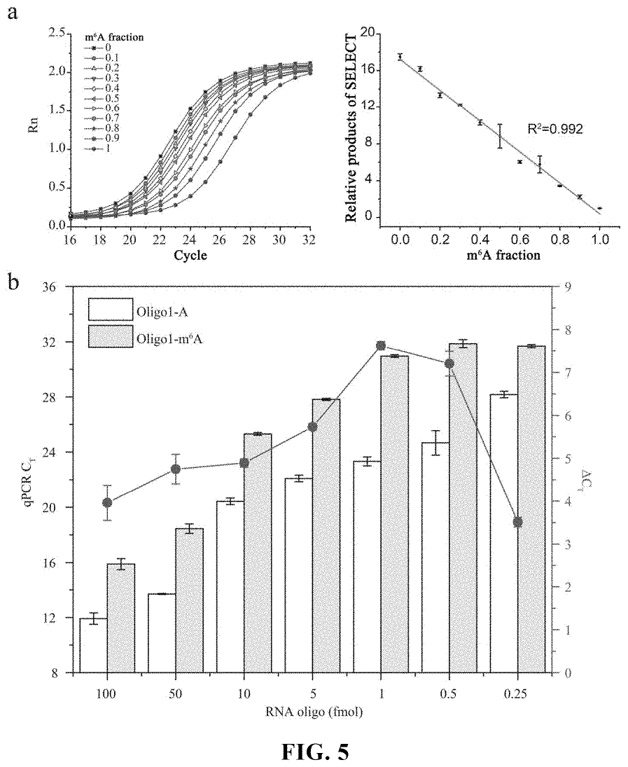

In order to accurately evaluate the performance of the SELECT method of the present application, Oligo1-m⁶A and Oligo1-A were mixed in the ratios of 0, 0.1, 0.2, 0.3, 0.4, 0.5, 0.6, 0.7, 0.8, 0.9, 1, respectively, and detected by SELECT method in combination with qPCR. FIG. 5a showed the linear relationship between the relative products of qPCR (2^{C}_{T} values normalized to the 2^{C}₁ value of 100% m⁶A) and m⁶A fraction in the sample. The experiment was repeated 3 times. Error bars, mean±s.d. Rn (normalized reporter) was the ratio of the fluorescence emission intensity of the fluorescent reporter group to the fluorescence emission intensity of the reference dye.

The SELECT method of the present application had a very high sensitivity, as shown in FIG. 5b, the SELECT method could distinguish A from m⁶A sites at target template concentrations ranging from 0.25 fmol to 100 fmol. The maximal ΔC_{T} of 7.62 cycles for a tested site X was observed for the 1 fmol RNA Oligo1 sample, suggesting that the selectivity of SELECT method for detecting m⁶A in RNA is up to 196.7-fold (2^{7.62}).

### Example 4 SELECT Method in Combination with qPCR for Detecting m⁶A Modification in Model m⁶A RNA Oligonucleotide

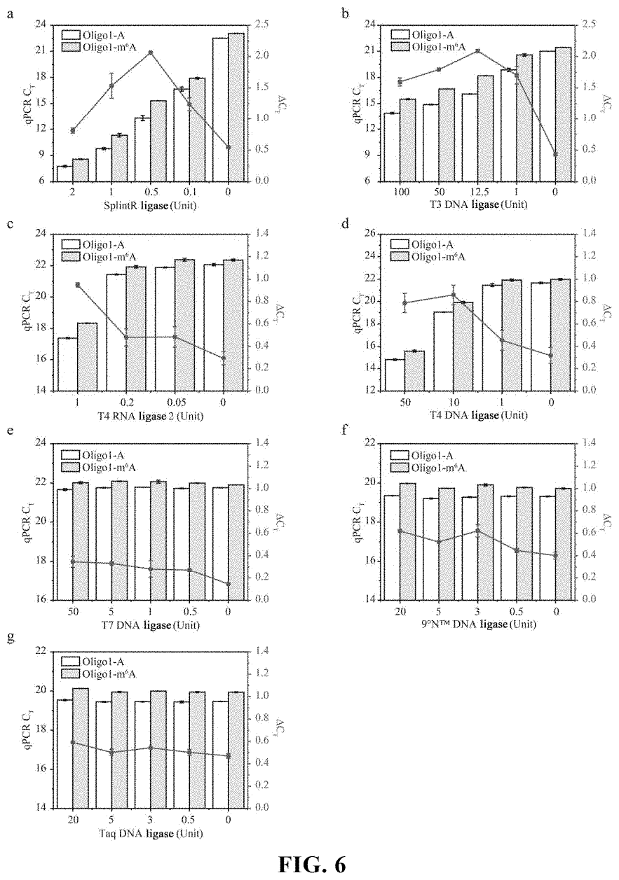

According to the method of step 6 in experimental methods, using the model oligonucleotide of Oligo1 (SEQ ID NO. 1) in Example 1 as a template, the performance of 7 ligases: SplintR ligase, T3 DNA ligase, T4 RNA ligase 2, T4 DNA ligase, T7 DNA ligase, 9° N DNA ligase, and Taq DNA Ligase were tested. The results were shown in FIG. 6, it can be seen that SplintR ligase, T3 DNA ligase, T4 RNA ligase 2 and T4 DNA ligase had a selectivity for m⁶A, among which SplintR ligase and T3 DNA ligase had a good selectivity, and SplintR ligase had a relative high efficiency for ligation and was suitable for the detection of low-trace sample.

### Example 5 SELECT Method Reaction Condition Test-1

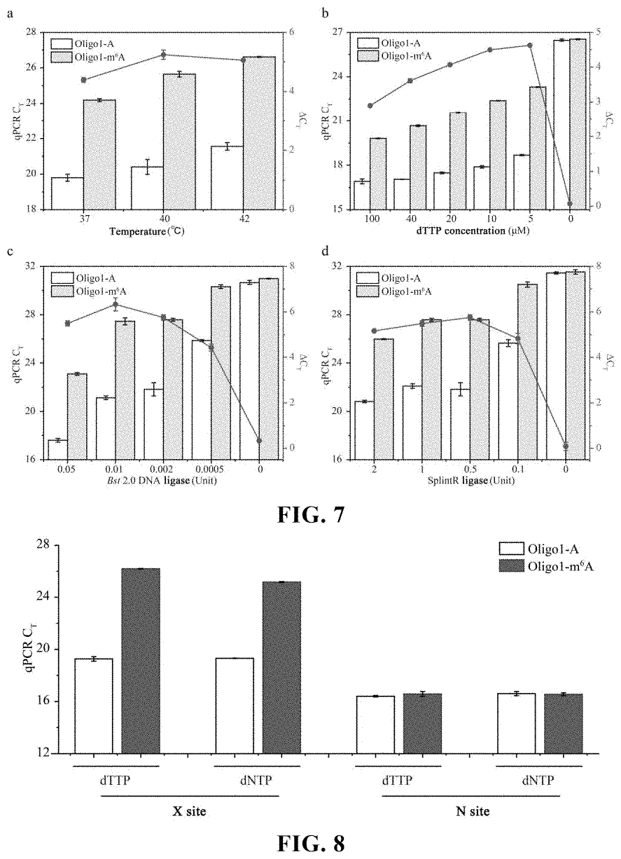

According to the method of Example 1, the present application expanded the reaction conditions for both the elongation and ligation steps, and settled on a simple one-tube reaction system. Specifically, this example tested the following reaction conditions: three reaction temperatures: 37° C., 40° C., and 42° C. (FIG. 7a); six concentrations of dTTP: 0, 5 μM, 10 μM, 20 μM, 40 μM, and 100 μM (FIG. 7b); five amounts of Bst 2.0 DNA polymerase: 0, 0.0005 U, 0.002 U, 0.01 U, and 0.05 U (FIG. 7c); and five amounts of SplintR ligase: 0, 0.1 U, 0.5 U, 1 U, and 2 U (FIG. 7d).

It can be seen from FIGS. 7a-d that the SELECT method exhibited a good selectivity for m⁶A at 37-42° C., 5-100 μM dTTP, 0.0005-0.05 U Bst 2.0 DNA polymerase, and 0.1-2 U SplintR ligase. The most preferable reaction conditions were: the reaction temperature of 40° C., 5 μM dTTP, 0.01 U Bst 2.0 DNA polymerase, and 0.5 U SplintR ligase.

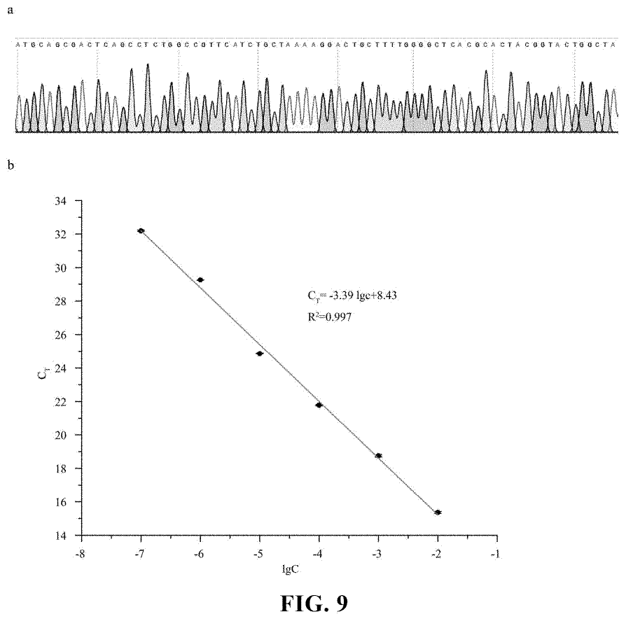

According to the method of Example 1, dTTP was replaced with dNTP, and it was found that dNTP could be used for the elongation step (see FIG. 8).

TA clone of the Oligo1-produced DNA fragments in pGEM-T vector were detected by SELECT method. The sequence of the Oligo1 qPCR amplicon was confirmed by Sanger sequencing (see FIG. 9a). The probe used for SELECT comprised two parts: qPCR adaptor and complementary strand of RNA template (melting temperature should exceed 50° C.). The PCR amplicons obtained by subjecting SEQ ID NO. 1 to SELECT detection with SEQ ID NO. 8 and SEQ ID NO. 9 were cloned into the pGEM-T vector, quantified by Nanodrop, and diluted stepwise (10-fold dilution for each step) to obtain standard samples, performed fluorescence quantitative PCR detection to plot a curve, and calculated adaptor amplification efficiency. The amplification specificity and efficiency of the primers targeting the site X were tested for the m⁶A status, the amplification efficiency of the designed qPCR primer was 97.2% calculated by the slope of −3.39, which confirmed that designed qPCR adaptor was sufficient for qPCR amplification (see FIG. 9b).

### Example 6 Reaction Condition Test of SELECT Method-2

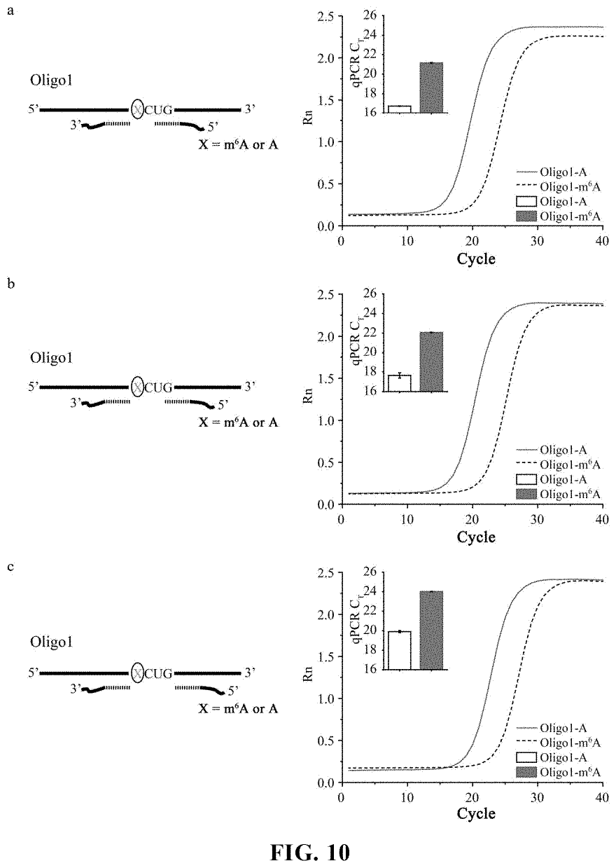

According to the method of Example 1, the present application designed more down probes: in which the first nucleotide of the 3′ terminal was complementary paired with the nucleotide located at a site with a distance of 2 nt, 3 nt and 4 nt from the RNA target site X at the downstream sequence of the RNA target site X. FIG. 10 showed these down probes could achieve good results for detection.

### Example 7 Verification of FTO Demethylation Activity

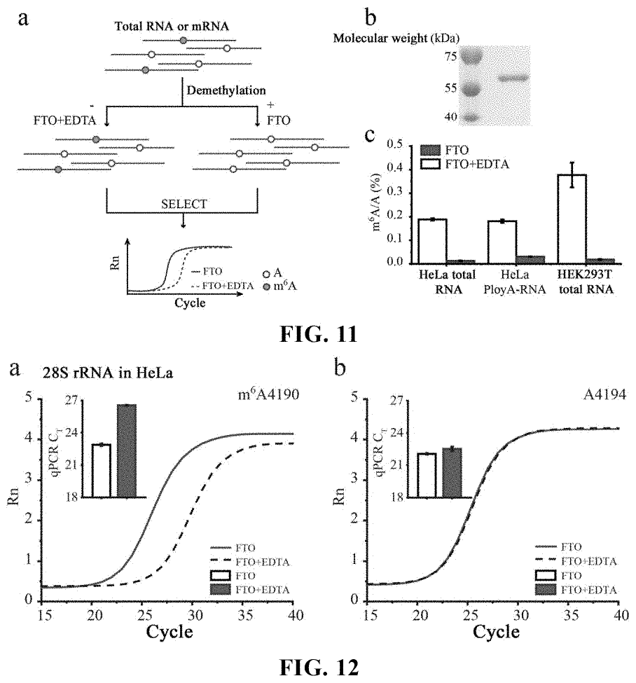

FTO was an m⁶A demethylase; it was Fe²⁺ and α-KG dependent, when EDTA was added to the reaction system to chelate free Fe²⁺, the m⁶A site could not be demethylated by FTO. FIG. 11a showed the process of FTO-assisted SELECT method for m⁶A detection. FIG. 11b showed the SDS-PAGE image of the coomassie blue staining of recombinant FTO protein purified from E. coli.

According to the method of step 1 of the experimental methods, the total RNA of HeLa cells and the total RNA of HEK293T cells, and the polyA-RNA of HeLa cells were extracted, respectively. The experimental group was treated with FTO, and the control group was treated with FTO+EDTA. The specific steps were as follows: for the experimental group: 40 μg total RNA or 2 μg polyA-RNA was mixed with FTO, 50 mM HEPES (pH 7.0), 2 mM L-ascorbic acid, 300 μM α-ketoglutarate (α-KG), 283 μM (NH₄)₂Fe(SO₄)₂.6H₂O and 0.2 U/μl RiboLock RNase inhibitor (purchased from Thermo Fisher Scientific), and reacted at 37° C. for 30 minutes. The reaction was quenched by adding 20 mM EDTA. For the control group: 20 mM EDTA was added before the demethylation reaction. The RNA was recovered by phenol-chloroform extraction and ethanol precipitation. FTO+EDTA-treated or FTO-treated samples were tested by the SELECT method described in step 3 of the experimental methods. The experiment was repeated 3 times, the error bars represented the mean±s.d.

FIG. 11c showed the FTO demethylation activity for m⁶A in total RNA or polyA-RNA isolated from HeLa or HEK293T cells. It can be seen that the levels of m⁶A in the total RNA of HeLa cells, polyA-RNA of HeLa cells and total RNA of HEK293T cells treated with FTO were significantly reduced: about 90% of the m⁶A sites were removed from the FTO-treated HeLa and HEK293T RNA samples, but not the FTO+EDTA cannot remove the m⁶A site.

### Example 8 FTO-Assisted SELECT Method for Detecting m⁶A Modifications in rRNA, lncRNA and mRNA

It should be noted that, 28S rRNA was detected by total RNA of HeLa cells, lncRNA MALAT1 was detected by polyA-RNA, and mRNA H1F0 was detected by total RNA of HEK293T cell.

The experimental group was treated with FTO, and the control group was treated with FTO+EDTA. The specific steps were as follows: for the experimental group: 40 μg total RNA or 2 μg polyA-RNA was mixed with FTO, 50 mM HEPES (pH 7.0), 2 mM L-ascorbic acid, 300 μM α-ketoglutarate (α-KG), 283 μM (NH₄)₂Fe(SO₄)₂.6H₂O and 0.2 U/μl RiboLock RNase inhibitor (purchased from Thermo Fisher Scientific), and reacted at 37° C. for 30 minutes. The reaction was quenched by adding 20 mM EDTA. For the control group: 20 mM EDTA was added before the demethylation reaction. The RNA was recovered by phenol-chloroform extraction and ethanol precipitation. FTO+EDTA-treated or FTO-treated samples were tested by the SELECT method described in step 3 of the experimental methods. In the SELECT method, the amounts of various RNAs were as follows: HeLa cells 28S rRNA, 30 ng, HeLa cells lncRNA MALAT1, 10 ng; HEK293T cells mRNA H1F0, 1 μg. m⁶A4190 and A4194 sites (input control) in HeLa cells 28S rRNA were detected; m⁶A2515 and A2511 (input control), as well as m⁶A2577, m⁶A2611 and A2614 sites (input control) in HeLa cells lncRNA MALAT1 were detected; and m⁶A1211 and A1207 sites (input control) in HEK293T cells mRNA H1F0 were detected. The experiment was repeated 3 times, the error represented the mean±s.d.

The combination of SELECT method and FTO demethylation step enabled clear identification of the known m⁶A4190 site present on 28S rRNA in HeLa (FIG. 12, left), and simultaneous analysis targeting a known non-m⁶A site (A4194, N site) on same rRNA, the input control N site showed no difference between the FTO-versus the FTO-EDTA-treated samples (FIG. 12, right).

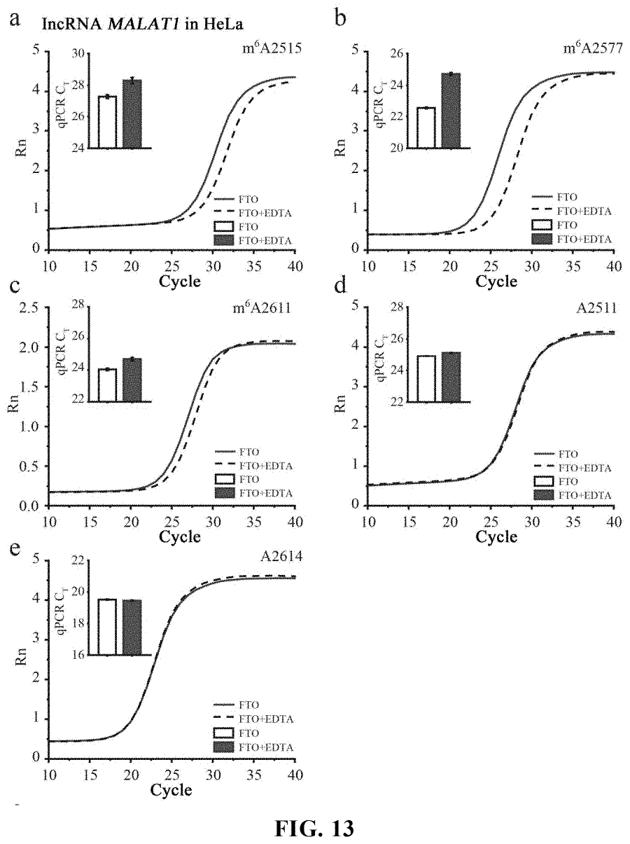

The combination of SELECT method and FTO demethylation enabled clear identification of three known m⁶A sites: m⁶A2515, m⁶A2577 and m⁶A2611 on the lncRNA MALAT1 transcript from HeLa cells; two non-m⁶A sites: A2511 and A2614 on the MALAT1 transcript for controlling the initial RNA input amount showed no difference between the FTO-versus the FTO-EDTA-treated samples (FIG. 13).

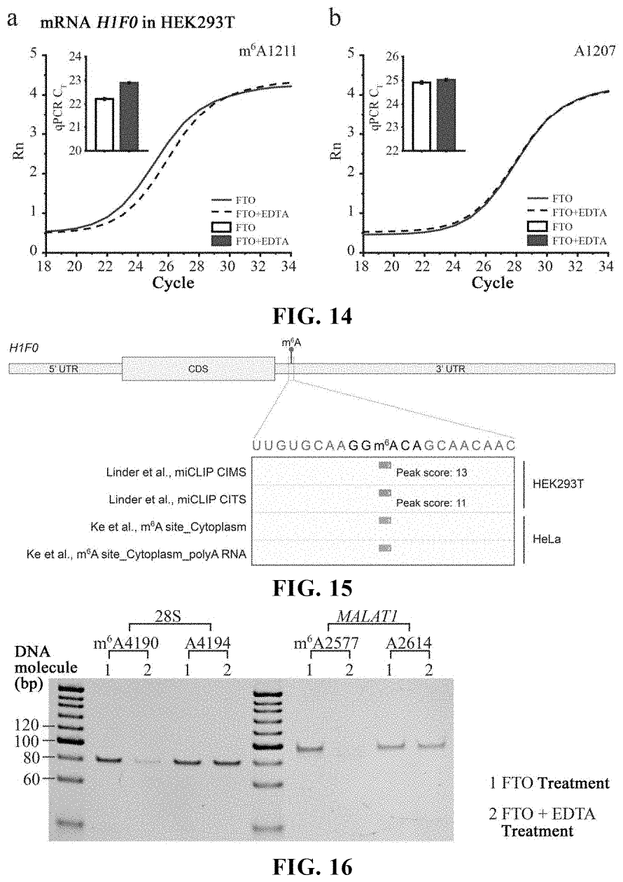

In addition to reconfirming the above known m⁶A sites, the combination of SELECT method of the present application and the FTO demethylation step was used to detect the presumed m⁶A sites on mRNA transcripts by the reported sequencing data of m⁶A from HEK293T and HeLa cells (the 1211 site in the 3′ UTR of H1F0, see FIG. 15). The results verified that the 1211 position in the mRNA of HEK293T cells was modified by m⁶A (FIG. 14). Thus, SELECT of the present application was an easy and highly effective method for precisely and efficiently identifying m⁶A sites on rRNA, lncRNA, and mRNA molecules from biological samples.

FTO-assisted SELECT method could also identify cellular m⁶A sites by PAGE analysis (see FIG. 16).

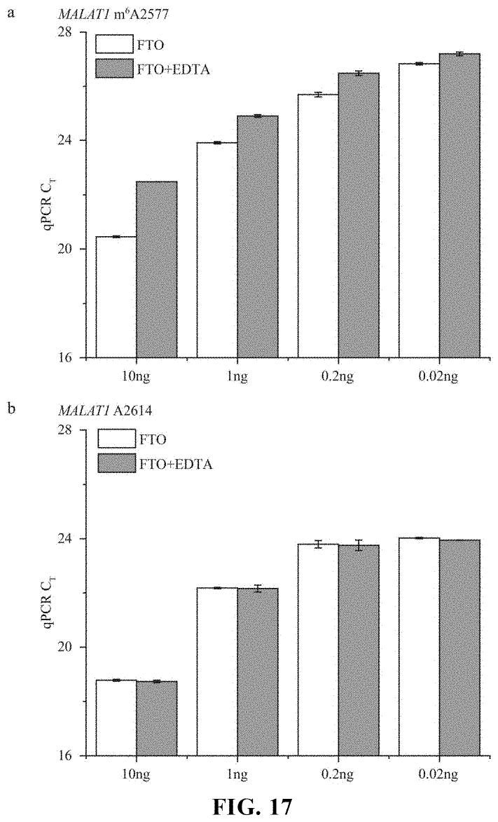

In addition, the detection limit of the input amount could be lowered to 0.2 ng of polyA-RNA (approximately 200-1400 cells) by using the method of this example (see FIG. 17).

### Example 9 SELECT Method for Quantifying the m⁶A Fraction in the Transcripts

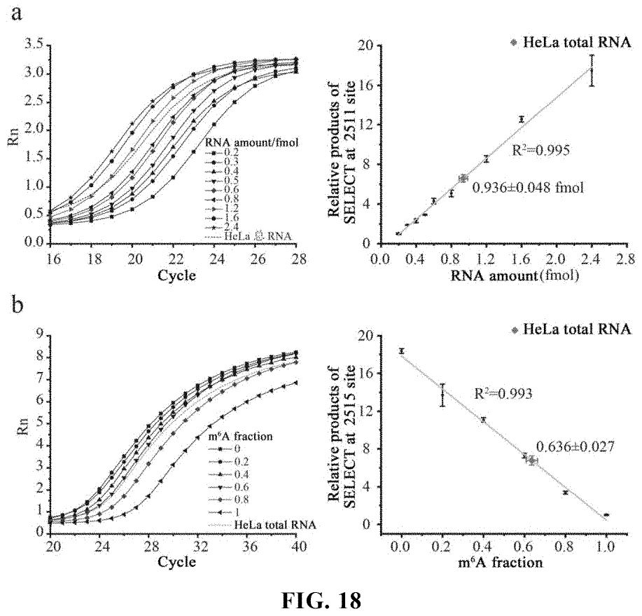

The SELECT method of the present application was also used to determine the m⁶A fraction of the m⁶A2515 site on MALAT1 lncRNA in HeLa. According to the sequence containing the 2488-2536 position of m⁶A2515 from HeLa cell MALAT1, an RNA of Oligo3 (SEQ ID NO. 3) consist of 49 nucleotides with an internal X site (in which X=m⁶A or A) was synthesized as a standard RNA. Firstly, different amounts of the standard RNA with either A, m⁶A, or a mixture were used to perform SELECT method in step 3 of the experimental methods at the A2511 site to generate a linear plot to quantify the amount of cellular MALAT1 transcript. The result showed that 3 μg of HeLa total RNA contained 0.936±0.048 fmol of MALAT1 transcripts (FIG. 18a). A series with 0.936 fmol of the standard RNA mixture with a known m⁶A fraction obtained by mixing Oligo3-m⁶A and Oligo3-A with 3 μg of HeLa total RNA were subjected to SELECT for analyzing the modification fraction at the MALAT1 m⁶A2515 site. 0.936 fmol of the standard RNA mixture with a different m⁶A fraction was used to run SELECT method in step 3 of the experimental methods at the m⁶A2515 site to generate a linear plot to quantify the absolute m⁶A fraction at the MALAT1 m⁶A2515 site in biological samples. The result showed that the m⁶A fraction at the MALAT1 m⁶A2515 site was 0.636±0.027 in HeLa (FIG. 18b). SCARLET et al. reported that the m⁶A fraction at the MALAT1 m⁶A2515 site was 0.61±0.03. Therefore, SELECT could precisely and conveniently determine the m⁶A fraction from total RNA.

FIG. 18 showed SELECT for determining the m⁶A fraction at the m⁶A2515 site of MALAT1 in HeLa. a) Quantification of MALAT1 transcripts in 3 μg of HeLa total RNA. A series of amount gradients of standard RNA (Oligo3) and 3 μg of HeLa total RNA were carried out for SELECT analysis at MALAT1 A2511 site. Real-time fluorescence amplification curves were shown in the left panel. The amount of MALAT1 transcripts calculated from the standard curve (right panel) was 0.936±0.048 fmol in 3 μg of HeLa total RNA. b) Quantification of the m⁶A fraction at the m⁶A2515 site of MALAT1 in HeLa. A series with 0.936 fmol of the standard RNA mixture with a known m⁶A fraction obtained by mixing Oligo3-m⁶A and Oligo3-A with 3 μg of HeLa total RNA were subjected to SELECT for analyzing the modification fraction at the MALAT1 m⁶A2515 site. Real-time fluorescence amplification curves were shown in the left panel, the right panel showed the m⁶A fraction at the MALAT1 2515 site calculated by the standard curve was 0.636±0.027 in HeLa cells. The error bars represented the mean±standard deviation. 2 biological replicates×2 technical replicates.

### Example 10 SELECT Method for Identifying the Biological Target Site of the m⁶A Modification Enzyme METTL3

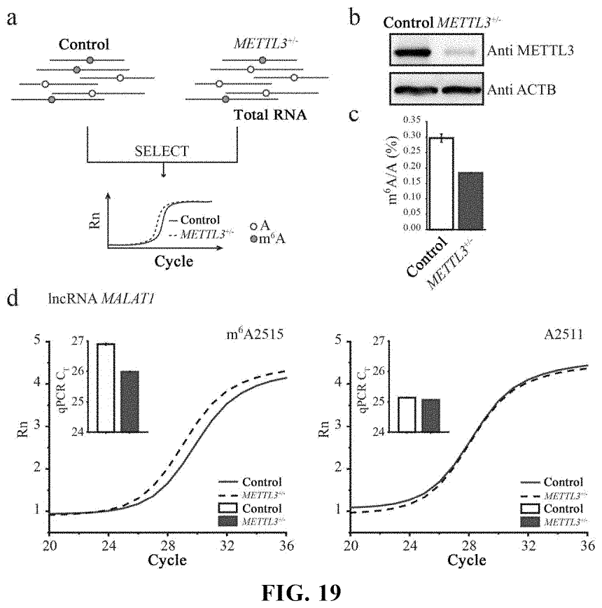

SELECT was also a powerful tool for functional studies of m⁶A metabolism because it can also be used in combination with genetics methods to confirm whether or not a particular m⁶A modification enzyme function to modify a specific m⁶A site. The m⁶A2515 site on MALAT1 lncRNA was used as a proof-of concept experimental system. It is reported that, two m⁶A modification enzyme METTL16 containing a catalytic subunit METTL3 could bind MALAT1 transcripts, but the enzyme responsible for the m⁶A modification of the 2515 site has not been confirmed. The CRISPR/Cas9 system was used to generate METTL3^{+/-} HeLa heterozygous cells in the present application; noted that homozygous METTL3^{+/-} cells were lethal. FIG. 19a showed that m⁶A was significantly reduced in METL3^{+/-} HeLa heterozygous cells compared to the control.

Western blotting confirmed that the heterozygous cells had reduced METTL3 levels by using anti-METTL3 antibodies (FIG. 19b). m⁶A was quantified by UPLC-MS/MS in step 9 of the experimental methods, the UPLC-MS/MS analysis showed that the total m6A levels in polyA-RNA were significantly lower in METTL3^{+/-} cells than in control cells (FIG. 19c). Subsequently, by using SELECT method in step 3 of the experimental methods, it was demonstrated that the extent of m⁶A modification at the 2515 site was significantly reduced in the METTL3^{+/-} cells compared to the control (FIG. 19d). Consistent with a specific role of METTL3 in methylating the 2515 site, no significant difference was observed in the amplification of the non-m⁶A A2511 site for controlling for initial RNA input amounts. Therefore, the 2515 site of MALAT1 was determined to be the biological target site of METTL3.

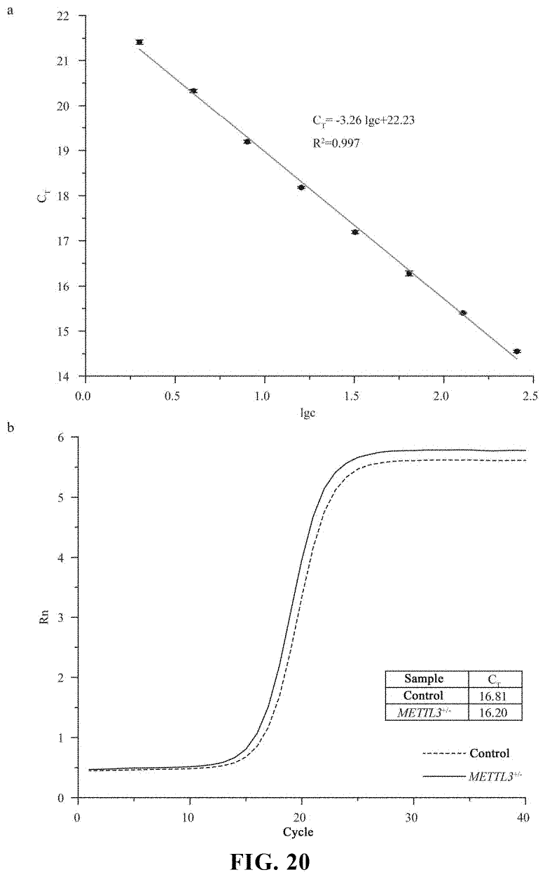

Note that m⁶A mediated mRNA degradation. To ensure that the total RNA from the control and METTL3^{+/-} cells loaded on SELECT contained equal amounts of MALAT1 transcripts, the inventors also performed qPCR analysis to adjust the amount of input total RNA (see FIG. 20).

### Example 11 SELECT Method for Detecting Other Types of RNA Modifications

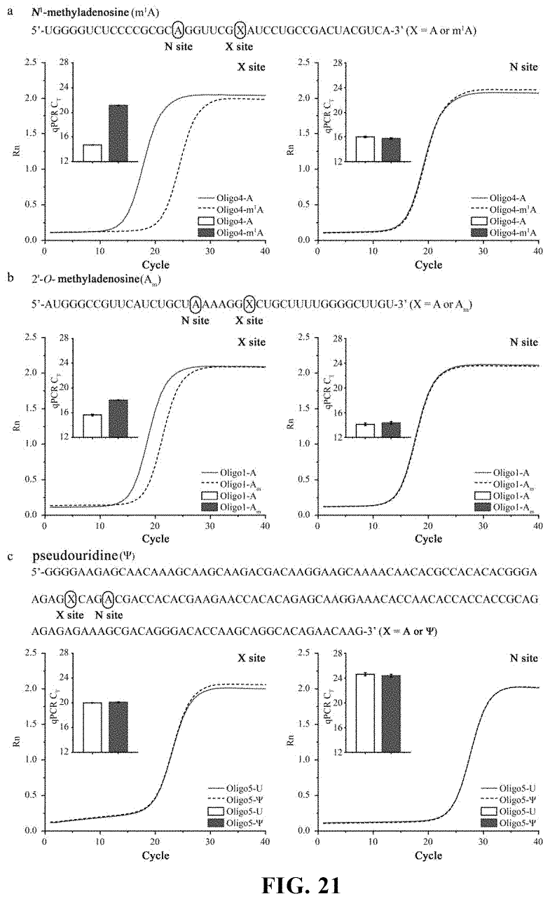

The inventors found that, by using the model oligonucleotides Oligo3 (SEQ ID NO. 3), Oligo4 (SEQ ID NO. 4), and Oligo5 (SEQ ID NO. 5) listed in Table 1 and the up probes and down probes listed in Table 3, the SELECT method in combination with the qPCR of Example 1 could effectively distinguish other RNA modifications, such as Ni-methyladenosine (m¹A) and 2′-O-methyladenosine (Am), but could not distinguish pseudouridine (ψ) (see FIG. 21).

The examples described above are only a part of the examples of the present application, not all of the examples. Based on the examples in the present application, all other examples obtained by those of ordinary skill in the art without creative work shall fall within the protection scope of the present application.

1. A method for detecting a chemical modification of an RNA target site X, comprising:
(1) obtaining an RNA sample, and selecting a target RNA segment containing an RNA target site X in the RNA sample;
(2) SELECT step: designing an up probe Px1 and a down probe Px2 for an upstream sequence and a downstream sequence of the RNA target site X within the target RNA segment, respectively, elongating the down probe Px2 through a DNA polymerase to obtain an elongated down probe Px2, and ligating the up probe Px1 and the elongated down probe Px2 through a ligase to obtain a SELECT product;
wherein, the up probe Px1 is complementary paired with the upstream sequence of the RNA target site X, and the first nucleotide of 5′-terminal of the up probe Px1 is complementary paired with a nucleotide located at a site with a distance of 1 nt from the RNA target site X at the upstream sequence of the RNA target site X;
the down probe Px2 is complementary paired with the downstream sequence of the RNA target site X, and the first nucleotide of 3′-terminal of the down probe Px2 is complementary paired with a nucleotide located at a site with a distance of 1, 2, 3, 4, 5, 6, 7, 8, 9 or 10 nt from the RNA target site X at the downstream sequence of the RNA target site X;
(3) PCR amplification step: performing PCR amplification of the SELECT product obtained in step (2), determining a threshold cycle of PCR or an amount of PCR amplification product; and
(4) comparing the threshold cycle of PCR to a threshold cycle of PCR reference, or comparing the amount of PCR amplification product to an amount of PCR amplification product reference, to determine if the target chemical modification is present at the RNA target site X.

2. The method according to claim 1, wherein the chemical modification is selected from the group consisting of m⁶A modification, m¹A modification, pseudouridine modification, and 2′-O-methylation modification.

3. The method according to claim 1, wherein the DNA polymerase is Bst 2.0 DNA polymerase or Tth DNA polymerase; and the ligase is selected from the group consisting of SplintR ligase, T3 DNA ligase, T4 RNA ligase 2, and T4 DNA ligase.

4. The method according to claim 1, wherein, in step (4), the threshold cycle of PCR reference is a threshold cycle of first PCR reference or a threshold cycle of second PCR reference, wherein:
the threshold cycle of first PCR reference is:
a threshold cycle of PCR of a first reference sequence determined by a method as same as that of the target RNA segment, wherein the first reference sequence comprises at least a nucleotide sequence II, and the nucleotide sequence II comprises a nucleotide sequence sharing a same nucleotide sequence with a nucleotide sequence I in the target RNA segment, wherein the nucleotide sequence I is a nucleotide sequence from a nucleotide that is complementary paired with the first nucleotide of 3′-terminal of the up primer of the site X to a nucleotide that is complementary paired with a nucleotide of 5′-terminal of the down primer of the site X in the RNA target segment, and no target chemical modification is present in an RNA target site X1 of the first reference sequence corresponding to the RNA target site X of the target RNA segment; or
the threshold cycle of second PCR reference is:
a threshold cycle of PCR of a second reference sequence determined by a method as same as that of the target RNA segment, wherein the second reference sequence comprises at least a nucleotide sequence II, and the nucleotide sequence II comprises a nucleotide sequence sharing a same nucleotide sequence with a nucleotide sequence I in the target RNA segment, wherein the nucleotide sequence I is a nucleotide sequence from a nucleotide that is complementary paired with a nucleotide of 3′-terminal of the up primer of the site X to a nucleotide that is complementary paired with a nucleotide of 5′-terminal of the down primer of the site X in the RNA target segment, and the target chemical modification is present in an RNA target site X2 of the second reference sequence corresponding to the RNA target site X of the target RNA segment.

5. The method according to claim 4, wherein:
when the threshold cycle of PCR is more than the threshold cycle of first PCR reference, it is determined that the target chemical modification is present in the RNA target site X; or
when the threshold cycle of PCR is equal to the threshold cycle of second PCR reference, it is determined that the target chemical modification is present in the RNA target site X.

6. The method according to claim 5, wherein, when the threshold cycle of PCR is at least 0.4-10 cycles more than the threshold cycle of first PCR reference, it is determined that the target chemical modification is present at the RNA target site X.

7. The method according to claim 1, wherein, in step (4), the amount of PCR amplification product reference is an amount of first PCR amplification product reference or an amount of PCR second amplification product reference, wherein:
the amount of first PCR amplification product reference is:
an amount of PCR amplification product of a first reference sequence determined by a method as same as that of the target RNA segment, wherein the first reference sequence comprises at least a nucleotide sequence II, and the nucleotide sequence II comprises a nucleotide sequence sharing a same nucleotide sequence with a nucleotide sequence I in the target RNA segment, wherein the nucleotide sequence I is a nucleotide sequence from a nucleotide that is complementary paired with a nucleotide of 3′-terminal of the up probe Px1 of the site X to a nucleotide that is complementary paired with a nucleotide at 5′-terminal of the down probe Px2 of the site X in the RNA target segment, and no target chemical modification is present in an RNA target site X1 of the first reference sequence corresponding to the RNA target site X of the target RNA segment; or
wherein, the amount of second PCR amplification product reference is:
an amount of PCR amplification product of a second reference sequence determined by a method as same as that of the target RNA segment, wherein the second reference sequence comprises at least a nucleotide sequence II, and the nucleotide sequence II comprises a nucleotide sequence sharing a same nucleotide sequence with a nucleotide sequence I in the target RNA segment, wherein the nucleotide sequence I is a nucleotide sequence from a nucleotide that is complementary paired with a nucleotide of 3′-terminal of the up probe Px1 of the site X to a nucleotide that is complementary paired with a nucleotide of 5′-terminal of the down probe Px2 of the site X in the RNA target segment, and the target chemical modification is present in an RNA target site X2 of the second reference sequence corresponding to the RNA target site X of the target RNA segment.

8. The method according to claim 7, wherein:
when the amount of PCR amplification product is less than the amount of first PCR amplification product reference, it is determined that the target chemical modification is present in the RNA target site X; or
when the amount of PCR amplification product is equal to the amount of second PCR amplification product reference, it is determined that the target chemical modification is present in the RNA target site X.

9. The method according to claim 1, the method further comprises following steps:
(c) controlling initial RNA input amounts, randomly selecting an RNA non-target site N in the target RNA segment; designing an up probe Pn1 and a down probe Pn2 for an upstream sequence and a downstream sequence of the RNA non-target site N, respectively, elongating the down probe Pn2 through a DNA polymerase to obtain an elongated down probe Pn2, and ligating the up probe Pn1 and the elongated down probe Pn2 through a ligase to obtain a SELECT product;
performing PCR amplification of the SELECT product, and determining a threshold cycle of FOR;
controlling the initial RNA input amounts of the target RNA segment according to the threshold cycle of PCR, so that the initial RNA input amounts of the target RNA segment is equal to initial RNA input amounts of a first reference sequence or a second reference sequence;
wherein,
the first reference sequence comprises at least a nucleotide sequence II, and the nucleotide sequence II comprises a nucleotide sequence sharing a same nucleotide sequence with a nucleotide sequence I in the target RNA segment, wherein when the site N is located upstream of the site X, the nucleotide sequence I is a nucleotide sequence from a nucleotide that is complementary paired with a nucleotide of 3′-terminal of the up probe Pn1 of the site N to a nucleotide that is complementary paired with a nucleotide of 5′-terminal of the down probe Px2 of the site X in the target RNA segment; when the site N is located downstream of the site X, the nucleotide sequence I is a nucleotide sequence from a nucleotide that is complementary paired with a nucleotide of 3′-terminal of the up probe Px1 of the site X to a nucleotide that is complementary paired with a nucleotide of 5′-terminal of the down probe Pn2 of the site N in the target RNA segment; and no target chemical modification is present in an RNA target site X1 of the first reference sequence corresponding to the RNA target site X of the target RNA segment; or
the second reference sequence comprises at least a nucleotide sequence II, and the nucleotide sequence II comprises a nucleotide sequence sharing a same nucleotide sequence with a nucleotide sequence I in the target RNA segment, wherein when the site N is located upstream of the site X, the nucleotide sequence I is a nucleotide sequence from a nucleotide that is complementary paired with a nucleotide of 3′-terminal of the up probe Pn1 of the site N to a nucleotide that is complementary paired with a nucleotide of 5′-terminal of the down probe Px2 of the site X in the target RNA segment, when the site N is located downstream of the site X, the nucleotide sequence I is a nucleotide sequence from a nucleotide that is complementary paired with a nucleotide of 3′-terminal of the up probe Px1 of the site X to a nucleotide that is complementary paired with a nucleotide of 5′-terminal of the down probe Pn2 of the site N in the target RNA segment; and target chemical modification is present in an RNA target site X2 of the second reference sequence corresponding to the RNA target site X of the target RNA segment.

10. The method according to claim 1, wherein the SELECT step is performed in a reaction system comprising:
an RNA sample;
dNTP;
a DNA polymerase;
a ligase.

11. The method according to claim 1, wherein the SELECT step is performed at a reaction temperature of 30-50° C.

12. The method according to claim 1, wherein the method further comprises following step prior to the step (1):
treating the RNA sample with an RNA demodification enzyme or a mixture of the RNA demodification enzyme and EDTA, respectively; wherein the RNA sample treated with the RNA demodification enzyme is used as a first reference sequence.

13. The method according to claim 1, wherein the RNA sample is total RNA, mRNA, rRNA, or lncRNA extracted from cells.

14. A method for identifying a target site of an RNA modification enzyme or an RNA demodification enzyme, comprising:
(1) preparing RNA modification enzyme—deficient or RNA demodification enzyme—deficient cells, or RNA modification enzyme—low expressed or RNA demodification enzyme—low expressed cells, culturing the cells and extracting an RNA after culturing the cells;
(2) determining a threshold cycle of PCR or an amount of PCR amplification product for an RNA target site X according to the steps (1)-(3) in the method of claim 1;
(3) comparing the threshold cycle of PCR with a threshold cycle of PCR reference, or comparing the amount of PCR amplification product with an amount of PCR amplification product reference, to determine if a chemical modification is performed by the RNA modification enzyme or the RNA demodification enzyme at the RNA target site X,
wherein, the threshold cycle of PCR reference is a threshold cycle of PCR for a normal cell determined by a method as same as that of the RNA modification enzyme—deficient or the RNA demodification enzyme—deficient cells, or the RNA modification enzyme—low expressed or the RNA demodification enzyme—low expressed cells,
the amount of PCR amplification product reference is an amount of PCR amplification product for the normal cell determined by a method as same as that of the RNA modification enzyme—deficient or the RNA demodification enzyme—deficient cells, or the RNA modification enzyme—low expressed or the RNA demodification enzyme—low expressed cells;
wherein the target site is a single gene-single site.

15. The method according to claim 14, wherein the RNA chemical modification is selected from the group consisting of m⁶A modification, m¹A modification, pseudouridine modification and 2′-O-methylation modification; the RNA chemical modification enzyme includes m⁶A modification enzyme.

16. A method for quantifying a RNA modification rate in transcripts, comprising:
(1) obtaining an RNA sample, and selecting a target RNA segment containing an RNA target site X in the RNA sample;
(2) determining an amount of the target RNA segment in the RNA sample, comprising:
(2a) randomly selecting an RNA non-target site N in the target RNA segment; designing an up probe Pn1 and a down probe Pn2 for an upstream sequence and a downstream sequence of the RNA non-target site N, respectively, elongating the down probe Pn2 through a DNA polymerase to obtain an elongated down probe Pn2, and ligating the up probe Pn1 and the elongated down probe Pn2 through a ligase to obtain a SELECT product; performing PCR amplification of the SELECT product, and determining a threshold cycle N of FOR;
(2b) gradient diluting a reference sequence to a series of concentrations, obtaining a threshold cycle Nn of PCR corresponding to each concentration by the method of step (2a), and determining a standard curve 1 according to the concentrations and the threshold cycle Nn of PCR;
wherein the reference sequence is a first reference sequence, a second reference sequence, or a mixture of the first reference sequence and the second reference sequence in any ratio,
the reference sequence comprises at least a nucleotide sequence II, and the nucleotide sequence II comprises a nucleotide sequence sharing a same nucleotide sequence with a nucleotide sequence I in the target RNA segment, wherein when the site N is located upstream of the site X, the nucleotide sequence I is a nucleotide sequence from a nucleotide that is complementary paired with a nucleotide of 3′-terminal of the up probe Pn1 of the site N to a nucleotide that is complementary paired with a nucleotide of 5′-terminal of the down probe Px2 of the site X in the target RNA segment, when the site N is located downstream of the site X, the nucleotide sequence I is a nucleotide sequence from a nucleotide that is complementary paired with a nucleotide of 3′-terminal of the up probe Px1 of the site X to a nucleotide that is complementary paired with a nucleotide of 5′-terminal of the down probe Pn2 at the site N in the target RNA segment,
and no target modification is present in an RNA target site X1 of the first reference sequence corresponding to the RNA target site X of the target RNA segment, and target chemical modification is present in an RNA target site X2 of the second reference sequence corresponding to the RNA target site of X of the target RNA segment;
(2c) comparing the threshold cycle N of PCR with the standard curve 1, and determining the amount of the target RNA segment in the RNA sample;
(3) mixing the first reference sequence and the second reference sequence in a series of molarity ratios to obtain a series of mixtures, and applying the (2) SELECT step and (3) PCR amplification step in the method of claim 1 to the mixtures to obtain a threshold cycle A1 of PCR or an amount A2 of PCR amplification product, determining a standard curve 2 according to the molarity ratios and the threshold cycle A1 of PCR or according to the molarity ratios and the amount A2 of PCR amplification product;
(4) applying the (2) SELECT step and (3) PCR amplification step in the method of claim 1 to the sample RNA to obtain a threshold cycle B1 of PCR or an amount B2 of PCR amplification product; and
(5) comparing the threshold cycle B1 of PCR or the amount B2 of PCR amplification product with the standard curve 2, to quantify the modification rate of the RNA target site X in the RNA sample.

17. The method according to claim 16, wherein the RNA sample is total RNA, mRNA, rRNA, or lncRNA extracted from cells.

18. The method according to claim 1, wherein a length of sequence of the up probe Px1 that is complementary paired with the upstream sequence of the RNA target site X is 15-30 nt; a length of sequence of the down probe Px2 that is complementary paired with the downstream sequence of the RNA target site X is 15-30 nt.

19. The method according to claim 1, wherein determining the threshold cycle of PCR is performed by qPCR fluorescence signal, or determining the amount of PCR amplification product is performed by polyacrylamide gel electrophoresis.

20. The method according to claim 3, wherein the DNA polymerase is Bst 2.0 DNA polymerase; the ligase is SplintR ligase or T3 DNA ligase.


SEQUENCE LISTING (54 sequences)

SEQ ID NO: 1 - RNA, 42 nt, Artificial Sequence
  oligo derived from modified HeLa RNA
  misc_feature (25)..(25): n is a or m6a or am
augggccguucaucugcuaaaaggncugcuuuuggggcuugu

SEQ ID NO: 2 - RNA, 42 nt, Artificial Sequence
  oligo derived from modified HeLa RNA
  misc_feature (25)..(25): n is a or m6a
augggccguucaucugcuaaaagancugcuuuuggggcuugu

SEQ ID NO: 3 - RNA, 49 nt, Artificial Sequence
  oligo derived from modified HeLa RNA
  misc_feature (28)..(28): n is a or m6a
aguagcuuaguuugaaaaaugugaaggncuuucguaacggaaguaauuc

SEQ ID NO: 4 - RNA, 42 nt, Artificial Sequence
  oligo derived from modified HeLa RNA
  misc_feature (24)..(24): n is a or m1a
uggggucuccccgcgcagguucgnauccugccgacuacguca

SEQ ID NO: 5 - RNA, 164 nt, Artificial Sequence
  oligo derived from modified HeLa RNA
  misc_feature (66)..(66): n is u or pseudouridine
ggggaagagcaacaaagcaagcaagacgacaaggaagcaaaacaacacgccacacacgggaagagncagacgaccacacgaagaaccacacagagcaaggaaacaccaacaccaccaccgcagagagagaaagcgacagggacaccaagcaggcacagaacaag

SEQ ID NO: 6 - DNA, 36 bp, Artificial Sequence
  Oligo1-X-T-upstream primer
tagccagtaccgtagtgcgtgagccccaaaagcagt

SEQ ID NO: 7 - DNA, 40 bp, Artificial Sequence
  Oligo1-X-downstream primer
ccttttagcagatgaacggccagaggctgagtcgctgcat

SEQ ID NO: 8 - DNA, 35 bp, Artificial Sequence
  Oligo1-X-downstream probe/ Oligo2-X-downstream probe
tagccagtaccgtagtgcgtgagccccaaaagcag

SEQ ID NO: 9 - DNA, 40 bp, Artificial Sequence; identical to SEQ ID NO: 7
  Oligo1-X-upstream probe

SEQ ID NO: 10 - DNA, 40 bp, Artificial Sequence
  Oligo2-X-upstream probe
tcttttagcagatgaacggccagaggctgagtcgctgcat

SEQ ID NO: 11 - DNA, 36 bp, Artificial Sequence; identical to SEQ ID NO: 6
  Oligo1-X-1-downstream probe

SEQ ID NO: 12 - DNA, 36 bp, Artificial Sequence; identical to SEQ ID NO: 6
  Oligo2-X-1-downstream probe

SEQ ID NO: 13 - DNA, 39 bp, Artificial Sequence
  Oligo1-X-1-upstream probe/ Oligo2-X-1-upstream probe
cttttagcagatgaacggccagaggctgagtcgctgcat

SEQ ID NO: 14 - DNA, 37 bp, Artificial Sequence
  Oligo1-X-2-downstream probe
tagccagtaccgtagtgcgtgagccccaaaagcagtc

SEQ ID NO: 15 - DNA, 37 bp, Artificial Sequence
  Oligo2-X-2-downstream probe
tagccagtaccgtagtgcgtgagccccaaaagcagtt

SEQ ID NO: 16 - DNA, 38 bp, Artificial Sequence
  Oligo1-X-2-upstream probe/ Oligo2-X-2-upstream probe
ttttagcagatgaacggccagaggctgagtcgctgcat

SEQ ID NO: 17 - DNA, 39 bp, Artificial Sequence
  Oligo1-X-4-downstream probe
tagccagtaccgtagtgcgtgagccccaaaagcagtcct

SEQ ID NO: 18 - DNA, 39 bp, Artificial Sequence
  Oligo2-X-4-downstream probe
tagccagtaccgtagtgcgtgagccccaaaagcagttct

SEQ ID NO: 19 - DNA, 36 bp, Artificial Sequence
  Oligo1-X-4-upstream probe/ Oligo2-X-4-upstream probe
ttagcagatgaacggccagaggctgagtcgctgcat

SEQ ID NO: 20 - DNA, 41 bp, Artificial Sequence
  Oligo1-X-6-downstream probe
tagccagtaccgtagtgcgtgagccccaaaagcagtccttt

SEQ ID NO: 21 - DNA, 41 bp, Artificial Sequence
  Oligo2-X-6-downstream probe
tagccagtaccgtagtgcgtgagccccaaaagcagttcttt

SEQ ID NO: 22 - DNA, 34 bp, Artificial Sequence
  Oligo1-X-6-upstream probe/ Oligo2-X-6-upstream probe
agcagatgaacggccagaggctgagtcgctgcat

SEQ ID NO: 23 - DNA, 34 bp, Artificial Sequence
  Oligo1-X+1-downstream probe
tagccagtaccgtagtgcgtgagccccaaaagca

SEQ ID NO: 24 - DNA, 41 bp, Artificial Sequence
  Oligo1-X+1-upstream probe
tccttttagcagatgaacggccagaggctgagtcgctgcat

SEQ ID NO: 25 - DNA, 36 bp, Artificial Sequence
  Oligo1-X+2-downstream probe
tagccagtaccgtagtgcgtgacaagccccaaaagc

SEQ ID NO: 26 - DNA, 42 bp, Artificial Sequence
  Oligo1-X+2-downstream probe
gtccttttagcagatgaacggccagaggctgagtcgctgcat

SEQ ID NO: 27 - DNA, 39 bp, Artificial Sequence
  Oligo4-X-downstream probe
tagccagtaccgtagtgcgtgtgacgtagtcggcaggat

SEQ ID NO: 28 - DNA, 35 bp, Artificial Sequence
  Oligo4-X-upstream probe
cgaacctgcgcggggcagaggctgagtcgctgcat

SEQ ID NO: 29 - DNA, 39 bp, Artificial Sequence
  Oligo4-X-7-downstream probe
tagccagtaccgtagtgcgtggtcggcaggattcgaacc

SEQ ID NO: 30 - DNA, 35 bp, Artificial Sequence
  Oligo4-X-7-upstream probe
gcgcggggagacccccagaggctgagtcgctgcat

SEQ ID NO: 31 - DNA, 38 bp, Artificial Sequence
  Oligo5-X-downstream probe
tagccagtaccgtagtgcgtgcttcgtgtggtcgtctg

SEQ ID NO: 32 - DNA, 36 bp, Artificial Sequence
  Oligo5-X-upstream probe
ctcttcccgtgtgtggcagaggctgagtcgctgcat

SEQ ID NO: 33 - DNA, 39 bp, Artificial Sequence
  Oligo4-X+4-downstream probe
tagccagtaccgtagtgcgtgtggttcttcgtgtggtcg

SEQ ID NO: 34 - DNA, 37 bp, Artificial Sequence
  Oligo4-X+4-upstream probe
ctgactcttcccgtgtgcagaggctgagtcgctgcat

SEQ ID NO: 35 - DNA, 39 bp, Artificial Sequence
  28S_m6A4190_downstream probe
tagccagtaccgtagtgcgtgcgccttaggacacctgcg

SEQ ID NO: 36 - DNA, 38 bp, Artificial Sequence
  28S_m6A4190_upstream probe
taccgtttgacaggtgtacagaggctgagtcgctgcat

SEQ ID NO: 37 - DNA, 39 bp, Artificial Sequence
  28S_A4194-downstream probe
tagccagtaccgtagtgcgtgagctcgccttaggacacc

SEQ ID NO: 38 - DNA, 39 bp, Artificial Sequence
  28S_A4194-upstream probe
gcgttaccgtttgacaggtcagaggctgagtcgctgcat

SEQ ID NO: 39 - DNA, 41 bp, Artificial Sequence
  MALAT1_m6A2515_downstream probe
tagccagtaccgtagtgcgtgaattacttccgttacgaaag

SEQ ID NO: 40 - DNA, 47 bp, Artificial Sequence
  MALAT1_m6A2515_upstream probe
ccttcacatttttcaaactaagctactcagaggctgagtcgctgcat

SEQ ID NO: 41 - DNA, 49 bp, Artificial Sequence
  MALAT1_m6A2577_downstream probe
tagccagtaccgtagtgcgtgggatttaaaaaataatcttaactcaaag

SEQ ID NO: 42 - DNA, 40 bp, Artificial Sequence
  MALAT1_m6A2577_upstream probe
ccaatgcaaaaacattaagtcagaggctgagtcgctgcat

SEQ ID NO: 43 - DNA, 45 bp, Artificial Sequence
  MALAT1_A2511_downstream probe
tagccagtaccgtagtgcgtgaattacttccgttacgaaagtcct

SEQ ID NO: 44 - DNA, 43 bp, Artificial Sequence
  MALAT1_A2511_upstream probe
cacatttttcaaactaagctactcagaggctgagtcgctgcat

SEQ ID NO: 45 - DNA, 40 bp, Artificial Sequence
  MALAT1_ m6A2611_downstream probe
tagccagtaccgtagtgcgtggtcagctgtcaattaatgc

SEQ ID NO: 46 - DNA, 50 bp, Artificial Sequence
  MALAT1_ m6A 2611-upstream probe
agtcctcaggatttaaaaaataatcttaaccagaggctgagtcgctgcat

SEQ ID NO: 47 - DNA, 41 bp, Artificial Sequence
  H1F0-m6A1211-downstream probe
tagccagtaccgtagtgcgtgcattagattggttgttgctg

SEQ ID NO: 48 - DNA, 38 bp, Artificial Sequence
  H1F0-m6A1211-upstream probe
ccttgcacaactggttaacagaggctgagtcgctgcat

SEQ ID NO: 49 - DNA, 37 bp, Artificial Sequence
  H1F0-A1207-downstream probe
tagccagtaccgtagtgcgtgtggttgttgctgtcct

SEQ ID NO: 50 - DNA, 39 bp, Artificial Sequence
  H1F0-A1207-upstream probe
gcacaactggttaaggaaacagaggctgagtcgctgcat

SEQ ID NO: 51 - DNA, 20 bp, Artificial Sequence
  qPCRF
atgcagcgactcagcctctg

SEQ ID NO: 52 - DNA, 21 bp, Artificial Sequence
  qPCRR
tagccagtaccgtagtgcgtg

SEQ ID NO: 53 - DNA, 21 bp, Artificial Sequence
  MALAT1_qPCRF
gacggaggttgagatgaagct

SEQ ID NO: 54 - DNA, 20 bp, Artificial Sequence
  MALAT1_qPCRR
attcggggctctgtagtcct